(19) United States
(12) Patent Application Publication
(10) Pub. No.: US 2008/0249606 A1
(43) Pub. Date: Oct. 9, 2008

(54) Methods for Determining the Effect of a Treatment on the Cross-Beta Structure Content of a Protein; Selection of Treatments and Uses Thereof

(76) Inventors: Martijn Frans Ben Gerard Gebbink, Eemnes (NL); Barend Bouma, Houten (NL)

Correspondence Address: TRASK BRITT, P.O. BOX 2550, SALT LAKE CITY, UT, 84110, US

(21) Appl. No.: 11/995,508

(22) PCT Filed: Jul. 13, 2006
(86) PCT No.: PCT/NL06/00365

§ 371 Date: Mar. 24, 2008

(30) Foreign Application Priority Data
Jul. 13, 2005 (EP) 05076614.6

(51) Int. Cl. G01N 33/68; A61F 2/82

(52) U.S. Cl. 623/1.15; 436/86; 435/7.1

(57) ABSTRACT

The invention relates to the field of biochemistry, biophysical chemistry, molecular biology, structural biology and medicine. More in particular, the invention relates to cross-β structure conformation. The invention provides a method for determining a difference in the cross-β structure content of a protein in a reference sample compared to said protein in a test sample wherein the test sample has been subjected to a treatment that is expected to have an effect on the cross-β structure content of said protein comprising—determining in said reference sample the cross-β structure content of said protein—subjecting said protein to a treatment that is expected to have an effect on the cross-β structure content to obtain said test sample—determining in said obtained test sample the cross-β structure content of said protein—determining whether the cross-β structure content of the reference sample is different from the cross-β structure content in the test sample.

The invention relates to the field of biochemistry, biophysical chemistry, molecular biology, structural biology and medicine. More in particular, the invention relates to cross-β structure conformation.

Refolding of polypeptides from their native fold into a conformation with an amyloid-like structure is an inherent property of proteinaceous molecules, independent of the amino-acids of which they are composed^{1,2}. Amyloids share a structural motif, termed the cross-β structure. We found that tissue-type plasminogen activator (tPA) and factor XII (FXII) are specifically activated by many polypeptides, once they have adopted the cross-β structure conformations. This led us to propose that a ‘cross-β structure pathway’ exists that regulates the recognition and clearance of unwanted proteins¹. Polypeptides can refold spontaneously, at the end of their life cycle, or refolding can be induced by environmental factors such as pH, glycation, oxidative stress, heat, irradiation, mechanical stress, proteolysis or contact with denaturing surfaces or compounds, such as negatively charged lipids, plastics or biomaterials. At least part of the polypeptide refolds and adopts the amyloid-like cross-β structure conformation. This cross-β structure containing conformation is then the signal that triggers a cascade of events that induces clearance and breakdown of the obsolete particle. When clearance is inadequate obsolete polypeptides can aggregate and form toxic structures ranging from soluble oligomers up to precipitating fibrils and amorphous plaques. Such cross-β structure containing structures underlie various diseases, such as Alzheimer's disease, Huntington's disease, diabetes mellitus type 2, systemic amyloidoses or Creutzfeldt-Jakob's disease, depending on the underlying polypeptide that accumulates and on the part of the body where accumulation occurs.

The presence of cross-β structures in proteins triggers multiple responses. As mentioned, cross-β structure comprising proteins can activate tPA and FXII, thereby initiating the fibrinolytic system and the contact system of hemostasis^{4,5}. Besides activation of the coagulation system through FXII, the cross-β structure conformation may induce coagulation, platelet aggregation and blood clotting via direct platelet activation and/or the release of tissue factor (Tf) by activated endothelial cells (described in more detail in a co-pending patent application). In addition, the complement system is another example of a proteolytic cascade that is activated by cross-β structure conformation. This system can be activated by the amyloid-β peptide associated with Alzheimer's Disease or by zirconium or aluminum or titanium. The latter being compounds that can induce cross-β structure conformation in proteins. The innate and adaptive immune systems are yet another example. Amyloid-β activates the innate and adaptive immune response⁶⁻⁸. β2-glycoprotein I is an auto-immune antigen only upon contact with a negatively charged lipid surface, such as cardiolipin⁹. We have now shown that cardiolipin induces cross-β structure conformation in β2-glycoprotein I (described in more detail in a co-pending patent application). Moreover, we have shown that ligands for Toll-like receptors that are implicated in the regulation of immunity induce cross-β structure conformation in proteins. These ligands include lipopolysaccharide and CpG oligodeoxynucleotides (ODN) (described in more detail in a co-pending patent application).

The β2-glycoprotein I protein (β2GPI), together with IgM antibodies, C1q and likely other proteins are all also acting in another way in the proposed cross-β structure pathway. It is assumed that a set of cross-β structure binding proteins bind specifically to sites of ‘danger’, e.g. negatively charged phospholipids, amyloid plaques, sites of ischemic injury, necrotic areas, all with its own specificity. Upon binding, the ‘dangerous’ condition is neutralized and for example excessive coagulation at negatively charged lipid surfaces will not occur. Secondly, the proteins bound to the ‘dangerous’ site undergo a conformational change resulting in the formation of the cross-β structure conformation. This fold then acts as a signal for cross-β structure binding proteins that are part of the ‘Cross-β structure pathway’, leading to the clearance of the bound protein or protein fragment and removal of the ‘danger’.

The Cross-β structure pathway may also act in yet another way. Proteins that circulate in complex with other proteins may comprise a shielded cross-β structure conformation. Once the protein is released from the accompanying protein, the cross-β structure becomes exposed, creating a binding site for cross-β structure binding proteins of the cross-β structure pathway. This may result in breakdown or clearance of the released protein. An example is factor VIII (FVIII), which circulates in complex with von Willebrand factor (vWF). In this complex, FVIII is prevented from clearance, so vWF may cover the clearance signal that becomes exposed after the complex is dissociated. This clearance signal is putatively the cross-β structure fold. Treatment of hemophilia patients with recombinant FVIII may induce inhibitors (anti-FVIII autoantibodies) because the patients lack sufficient vWF to shield the clearance signal comprising the cross-β structure conformation. Excess exposure of FVIII comprising cross-β structure conformation may induce activation of the immune system and generation of anti-FVIII antibodies similar to the generation of anti-β2GPI autoimmune antibodies by β2GPI bound to negatively charged phospholipids and possibly autoimmune responses.

As a more detailed embodiment homeostasis is discussed in more detail. Homeostasis is threatened by an array of foreign factors that, when introduced to the circulation, or exposed to the circulation, or exposed to cells aligning the circulation, can cause thrombotic, inflammatory and/or immunogenic complications. Such factors include, but are not limited to, micro-organisms, extra-corporal circulation devices, kidney dialysis devices, stents, valves, and implants composed of for example biomaterials, metals, plastics or combinations thereof.

FXII can be activated by negatively charged agents. For example, when blood is drawn into a glass tube it rapidly clots, due to activation of FXII. However, when the tube is made of plastic clotting is delayed. This mechanism of this contact system of coagulation is termed the intrinsic pathway because all clotting factors are present in plasma; in contrast to the extrinsic pathway, which requires the presence of tissue factor on the surface of cells, that is not exposed to the circulation during homeostasis. Interestingly, the nature of the FXII activator in vivo is still unknown. We now found that cross-β structure, that is formed when globular proteins unfold due to any denaturing trigger, is a trigger for FXII and contact activation. Since negatively charged surfaces, such as glass, induce denaturation of proteins, it may well be possible that activation of FXII is secondary to formation of cross-β structure by negatively charged surfaces. We have tested whether activation of FXII by dextran sulphate 500,000 Da (DXS500k) and kaolin is accompanied and mediated by cross-β structure, and our results indeed show that this is occurring. We have determined that plasma exposure to a surface of DXS500k or kaolin indeed induces cross-β structure conformation by staining with Thioflavin T (ThT) and by binding of a recombinant finger domain. In addition, we will test whether the amyloid binding reagents Congo Red, ThT, recombinant finger domains of tPA, FXII, HGFA and fibronectin, or full-length tPA, FXII, HGFA, fibronectin, serum amyloid P component (SAP), anti-cross-β structure antibodies and/or a soluble fragment of receptor for advanced glycation end-products (sRAGE) inhibit activation of FXII induced by DXS500k, kaolin, any other activating surface, or by denatured polypeptides comprising the cross-β structure conformation.

tPA is a serine protease involved in fibrin clot lysis. tPA stimulates activation of plasminogen into plasmin. Fibrin serves as an efficient cofactor in stimulating tPA mediated plasmin formation. Besides fibrin and fibrin fragments a large number of other proteins or protein fragments have been found that stimulate tPA activity, though that exhibit no apparent amino-acid sequence homology. Therefore, the anticipated common structural basis underlying the acquired tPA binding remained elusive. We recently found that the amyloid-like cross-β structure conformation, the structural element found in amyloid deposits in diseases such as Alzheimer's disease, is a prerequisite and the common denominator in tPA-binding ligands^{1,3}. FXII shows close homology with tPA and is known to be activated by amyloid-β (Aβ) and by bacteria with an amyloid core¹⁰. The domain structure of FXII includes, like tPA, a finger domain and its sequence shows the closest homologies with tPA. FXII also binds fibrin (Sanchez et al. 2003, ISTH XIX Congress; surface deposited fibrin activates FXII and the intrinsic coagulation pathway) and FXII can also, like tPA, mediate the conversion of plasminogen to plasmin^{11,12}. We found that FXII, like tPA, is activated by polypeptides with amyloid-like cross-β structure conformation in general. Moreover, we established that well-known activators of FXII, DXS500k and kaolin, induce amyloid-like cross-β structure conformation in proteins and that DXS500k is only then an effective activator of FXII when an excess of protein cofactor over the amount of FXII present is added to the reaction mixture. Thus, in contrast to direct activation by binding to negatively charged surfaces, FXII is activated by (plasma) proteins that denature and form amyloid on negatively charged surfaces, or denature by any other means, e.g. pH change, exposure to radicals, proteolysis, glycation, oxidation, change in temperature. It is thus stated that the cross-β structure conformation regulates contact activation and fibrinolysis.

At present, it is assumed that activation of FXII directly involves binding to negatively charged surfaces. Based on our findings, we show that negatively charged surfaces induce amyloid cross-β structure formation and that this structure element triggers FXII activation. This finding renews the view on contact-mediated activation of blood coagulation.

We further disclose that formation of cross-β structure underlies a variety of complications associated with the use of therapeutics, such as protein therapeutics or constituents thereof. More specifically it is disclosed that devices or materials used to prepare said therapeutics can mediate the formation of cross-β structure in said protein or said therapeutic or any of its constituents. Even more specifically it is disclosed that biocompatible materials, preferably used in a subject, for example for dialysis or for delivery of a compound, preferably a therapeutic to a subject, can induce formation of cross-β structure. Said complications include but are not limited to thrombotic complications, inflammatory responses, bleeding, coagulation, or immunogenicity.

Based on these findings we developed, amongst other methods, methods for testing the effect of a certain condition on the cross-β structure content of a protein. Such a method is for example extremely useful and of utmost importance in determining the biocompatibility of materials. More detailed examples and uses are provided below.

In a first embodiment, the invention provides a method for determining a difference in the cross-β structure content of a protein in a reference sample compared to said protein in a test sample wherein the test sample has been subjected to a treatment that is expected to have an effect on the cross-β structure content of said protein comprising

determining in said reference sample the cross-β structure content of said protein

subjecting said protein to a treatment that is expected to have an effect on the cross-β structure content to obtain said test sample

determining in said obtained test sample the cross-β structure content of said protein

determining whether the cross-β structure content of the reference sample is different from the cross-β structure content in the test sample.

A cross-β structure is defined as a part of a protein or peptide, or a part of an assembly of peptides and/or proteins, which comprises an ordered group of β-strands; typically a group of β-strands arranged in a β-sheet, in particular a group of stacked β-sheets, also referred to as “amyloid”. A typical form of stacked β-sheets is in a fibril-like structure in which the β-sheets may be stacked in either the direction of the axis of the fibril or perpendicular to the direction of the axis of the fibril. Of course the term peptide is intended to include oligopeptides as well as polypeptides, and the term protein includes proteins with and without post-translational modifications, such as glycosylation. It also includes lipoproteins and complexes comprising proteins, such as protein-nucleic acid complexes (RNA and/or DNA), membrane-protein complexes, etc. A β-sheet is a secondary structural element in a peptide and/or protein. A cross-β structure comprises a tertiary or quaternary structural element in a peptide and/or protein and can be formed upon for example denaturation, proteolysis, chemical modification or unfolding of proteins. Said cross-β structure is generally absent in non-altered globular proteins. Said cross-β structure is in general composed of stacked β-sheets. In a cross-β structure the individual β-strands run either perpendicular to the long axis of a fibril, or the β-strands run in parallel to the long axis of a fibril. In some cases, the direction of the stacking of the β-sheets in cross-β structures is perpendicular to the long axis of the fibril¹. Moreover, if it is determined that a compound binds to a cross-β structure in a protein, such a determined cross-β structure binding compound can further be used in the detection of other proteins that comprise a cross-β structure. The proteins that are detected by such a method are also included by the term cross-β structure.

The term cross-β structure, cross-β structure conformation and cross-β conformation will be used herein interchangeably.

We have observed that the hexapeptide FP6 can form oligomers consisting of up to 15 peptide molecules, with cross-β structure conformation. Various preparations exhibit different tPA activating properties, appear differently on TEM images, enhance Congo red fluorescence differently and have formed distinct cross-β structure conformations, as depicted from X-ray diffraction data sets. These data provide insight in the diverse nature of the cross-β structure conformation. In fact, the cross-β structure conformation, also referred to as β-pleated sheets, cross-β sheets or cross-β spine, is an ensemble of structures. Polypeptides differing in amino-acid sequence or length, or a polypeptide treated in different ways, may appear with cross-β structures that differ from each other to some extent.

The difference in the cross-β structure content of a protein in a reference sample compared to the cross-β structure content of a protein in a test sample reflects the effect of the treatment that is expected to have an effect on the cross-β structure content of said protein and can go various ways. For example, the test sample comprises a protein that has a higher cross-β structure content compared to said protein in the reference sample and hence the treatment has cross-β structure inducing capabilities/effects. Or the test sample comprises a lower cross-β structure content compared to the reference sample and the treatment thus masks the cross-β structures present in the reference sample and/or is capable of removing cross-β structures from a protein and/or is capable of inducing refolding back from a cross-β structure conformation to a different protein fold and/or is capable of removing molecules with cross-β structure conformation. Or the test sample comprises a different type of cross-β structure fold compared to the reference sample and the treatment thus induces structural rearrangements in the cross-β structure fold that was originally present. Any combination of the aforementioned possibilities may also occur. The embodiment in which the cross-β structure content of a protein essentially remains the same, i.e. does essentially not change/increase/decrease, is described in more detail hereunder. Preferably, the structure of a protein (comprising no detectable cross-β structures) essentially remains the same, i.e. no cross-β structures are formed.

Preferably, the reference sample and the test sample are one and the same sample which for example both originate from a (larger) batch of protein comprising cross-β structure conformation wherein one part of said batch is used to determine a reference value/point and/or a standard curve and another part of said batch is subjected to a treatment expected to have an effect on the cross-β structure content of said protein to obtain a test sample of which subsequently the cross-β structure content of said protein is determined. Even more preferred a sample is first used to determine the cross-β structure content and the same sample (or a part thereof) is subsequently subjected to the treatment expected to have an affect on the cross-β structure content to obtain a test sample. This is for example accomplished by the use of appropriate standard curves which are measured before and after the treatment.

The method according to the invention can be performed qualitatively as well as quantitatively and hence reference to cross-β structures content of a protein or reference/test value or point is herein defined as to cover both a quantitative assay as well as a qualitative assay.

The step in which a protein is subjected to a treatment expected to have an effect on the cross-β structure content of said protein can be performed in different ways and largely depend on the type of the to be tested treatment. If one for example wants to determine the effect of the pH of a buffer on the cross-β structure content of a protein, said protein is dissolved in or brought into contact with or diluted with buffers with different pH-values. After certain incubation time (which depend on the purpose; if one wants to determine the effect of a long term storage buffer, the incubation time will be longer compared to a situation in which one want to test the short-term effect of a buffer) the cross-β structure content of said protein in the different buffers is determined and compared. If one for example wants to determine the effect of surfaces on the cross-β structure content one can incubate a protein with a solid surface or pass said protein along said surface (for example by creating a flow of a solution comprising a protein along said surface). It is clear to a skilled person that the particular type of subjecting conditions depends largely on the particular to be tested treatment.

As described above, a particular useful embodiment is a method for selecting a circumstance that does not induce cross-β structure conformation in a protein or that does not change the cross-β structure content of a protein. Such a circumstance can then be used to for example prolong the activity of a certain protein that would be lost when the protein refolds into cross-β structure conformation. Moreover, prevention of cross-β structures formation results in decrease and preferably in completely preventing immunogenic and/or thrombogenic and/or inflammatory responses. Hence, in a preferred embodiment the invention provides a method for selecting a treatment that essentially preserves the structure of a protein comprising

determining in a reference sample the cross-β structure content of said protein

subjecting said protein to a treatment that is expected to have an effect on the cross-β structure content to obtain a test sample

determining in said test sample the cross-β structure content of said protein

selecting the treatment that essentially preserves the structure of said protein. In yet another preferred embodiment, the invention provides a method for selecting a treatment that essentially preserves the cross-β structure content of a protein comprising

determining in a reference sample the cross-β structure content of said protein

subjecting said protein to a treatment that is expected to have an effect on the cross-β structure content to obtain a test sample

determining in said test sample the cross-β structure content of said protein

selecting the treatment that essentially preserves the cross-β structure content of said protein.

In such embodiments the structure (preferably comprising no detectable cross-β structure) remains the same upon said treatment or the cross-β structure content of a protein is essentially the same (at least not qualitatively or quantitatively different) in the reference sample and the test sample, i.e. the tested treatment has essentially no influence on the cross-β structure content of a protein. The identification of such a treatment opens up new possibilities in respect of protein uses, storage, quality control etc. If one for example determines that biocompatible material A preserves the (cross-β) structure (fold) of a blood protein (and preferably all blood proteins), said biocompatible material may advantageously be used in the design of dialysis apparatus or as a storage means for blood. Hence, such a method is very useful for manufactures of biocompatible material (that comes in contact with for example blood proteins) and moreover immunogenic and/or thrombogenic and/or inflammatory responses in reaction to said biocompatible material will be decreased or more preferably completely absent. More uses and applications will be discussed later on.

The sample (or the to be tested) protein can take different forms. For example, said protein may be in a dried, solid form and the to be tested treatment comprises different reconstitution buffers or different storage conditions (for example different humidity conditions and the effect of said humidity on for example the activity of said protein). In a preferred embodiment, said protein is a protein in a solution. In an even more preferred embodiment said solution is a body fluid, such as blood or lymph fluid, or cerebrospinal fluid or synovial fluid or a part derived thereof (for example plasma). In yet another preferred embodiment the protein is part of a cell (for example a surface protein) or a constituent of tissue or an extracellular matrix protein. In case the protein is part of a more solid sample, said sample may further be subjected to a homogenization step.

Said protein (in solution or as part of a cell, either or not in tissue or in matrix) may be a single type of protein or a mixture of proteins (possibly in solution). Detection of single types of proteins and mixtures of protein is described in more detail later on.

In a preferred embodiment the invention provides a method according to the invention wherein one particular protein and one particular treatment is tested. For example, testing the effect of a certain storage conditions (for example temperature) on the cross-β structure content of one particular pharmaceutical protein. For example, erythropoetin is used as a pharmaceutical to increase in a subject in need thereof, amongst others, the amount of red blood cells. Subjecting said pharmaceutical composition comprising erythropoetin to different storage temperatures and determining the cross-β structure content of said differently treated samples gives insight into the most appropriate storage temperature. Thus, in a particular preferred embodiment only one parameter is changed.

A method according to the invention involves a step wherein the cross-β structure content of protein is determined. Such a step generally comprises the use of a cross-β structure binding compound. Examples of cross-β structure binding compounds are described in Table 1 or 2 or 3.

The compounds listed in Table 1 and the proteins listed in Table 2 all bind to polypeptides with a non-native fold. In literature, this non-native fold has been designated as protein aggregates, amyloid, amyloid oligomers, cross-β structure, β-pleated sheet, cross-β spine, denatured protein, cross-β sheet, β-structure rich aggregates, amorphous/proteinaceous plaque, tangle, infective aggregating form of a protein, unfolded protein, amyloid-like fold/conformation and perhaps alternatively. The common theme amongst all polypeptides with a non-native fold, that are ligands for one or more of the compounds listed in Table 1 and 2, is the presence of a cross-β structure conformation.

The compounds listed in Table 1 and 2 are considered to be only a subset of all compounds known to day to bind to non-native protein conformations. The lists are thus non-limiting. More compounds are known today that bind to amyloid-like protein conformation. For example, in patent AU2003214375 it is described that aggregates of prion protein, amyloid, and tau bind selectively to polyionic binding agents such as dextran sulphate or pentosan (anionic), or to polyamine compounds such as poly (Diallyldimethylammonium Chloride) (cationic). Compounds with specificity for non-native folds of proteins listed in this patent and elsewhere are in principle equally suitable for methods and devices disclosed in this patent application. Moreover, also any compound or protein related to the ones listed in Table 1 and 2 are covered by the claims. For example, point mutants, fragments, recombinantly produced combinations of cross-β structure binding domains and deletion- and insertion mutants are part of the set of compounds as long as they are capable of binding to a cross-β structure (i.e. as long as they are functional equivalents). Even more, also any newly discovered small molecule or protein that exhibits affinity for the cross-β structure fold can in principle be used in any one of the methods and applications disclosed herein.

The compounds listed in Table 3 are also considered to be part of the ‘Cross-β structure pathway’, and this is based on literature data that indicates interactions of the listed molecules with compounds that likely comprise the cross-β structure fold but that have not been disclosed as such. For example, scavenger receptor MARCO binds to acetylated low-density lipoprotein and to bacteria. We showed that protein modifications oxidation and glycation introduces the cross-β structure fold in proteins¹ and we pointed to a role for the amyloid core structures of bacteria in the interactions with a host¹⁰.

The step of determining the cross-β structure content generally comprises the immobilisation of a compound as described in Table 1 or 2 or 3 on a solid surface followed by contacting a sample (either or not exposed to a treatment that is expected to have an effect on the cross-β structure content) with said immobilised cross-β structure binding compound and detection of the bound cross-β structure comprising protein with (another) cross-β structure binding compound (for example obtained from Table 1 or 2 or 3) or via specific detection of the cross-β structure comprising protein.

A suitable sandwich ELISA is described in more detail and comprises the following steps: (i) Immobilisation of any of the compounds of Table 1 or 2 or 3 on a carrier, (ii) Incubation of a reference sample and/or test sample and/or optionally a standard curve with a compound with known cross-β structure conformation, (iii) Performing one or multiple wash step(s), (iv) Incubation with a second cross-β structure binding compound, (v) And finally qualify or quantify. Alternatively, the amount of bound protein can be quantified by using an antibody/ligand/substrate specific for the protein(s) of the sample that is either or not exposed to a putatively denaturing condition (i.e. the treatment that is expected to have an effect on the cross-β structure content). In an alternative way, any of the compounds in Table 1 or 2 or 3 can be immobilized on beads. The solid support with immobilized cross-β structure binding compound can be integrated in any flow device. For example in a surface plasmon resonance apparatus. When the putatively denaturing condition is a solid surface, this surface before and after contacting with a protein sample, can be washed and exposed to a mixture of tPA and plasminogen, preferably at 37° C., preferably in HBS (10 mM HEPES, 4 mM KCl, 137 mM NaCl, pH 7.3), preferably with swirling. After an incubation time of preferably 1-3 h, the tPA-plasminogen solution can be transferred to an ELISA plate, plasmin substrate S-2251 added and the amount of generated plasmin determined, with the use of standard curves with a compound with cross-β structure conformation.

As an example, in complex mixtures, the cross-β structure content of each individual protein can be assessed by contacting the mixture to, for example, a solid surface with an immobilized cross-β structure binding compound, followed by an isolation step and a washing step, finalized by contacting the solid surface with an immobilized cross-β structure binding compound and putatively various bound proteins, individually with antibodies specific for the putatively various bound proteins, that comprise cross-β structure conformation.

In a preferred embodiment, the invention provides a method wherein at least one of said determining steps is performed with an enzymatic assay. An example of such an assay is described in more detail. Such an enzymatic assay comprises the use of tPA and plasminogen and plasmin substrate, preferably S-2251 (Chromogenix Spa, Milan, Italy), in a suitable buffer, preferably the buffer is HBS (10 mM HEPES, 4 mM KCl, 137 mM NaCl, pH 7.3). Such an assay further comprises a standard curve with a control with cross-β structure conformation and titration curve with a sample before and after a treatment/exposure to a putatively denaturing condition. In an alternative assay use is made of FXII with activated FXII substrate, preferably S-2222 or S-2302 in a suitable buffer; preferably, the buffer contains 50 mM, 1 mM EDTA, 0.001% v/v Triton-X100. Standard curves with known cross-β structure rich activators of FXII; preferably DXS500k with a protein; preferably the protein is endostatin or albumin; preferably with glycated haemoglobin, Aβ, amyloid fibrin peptide NH₂-148KRLEVDIDIGIRS160-COOH with K157G mutation. In yet another alternative assay use is made of FXII with prekallikrein and high molecular weight kininogen and either substrate Chromozym-PK for kallikrein or a substrate for activated FXII in a suitable buffer; preferably HBS. Standard curves with known cross-β structure rich activators of FXII; preferably DXS500k or kaolin with a protein; preferably the protein is endostatin or albumin; preferably with glycated haemoglobin, Aβ, amyloid fibrin peptide NH₂-148KRLEVDIDIGIRS160-COOH with K157G mutation.

However, it is also possible to determine the effect of, for example a solid surface by contacting said solid surface with a protein and determine whether any cross-β structure is present at said solid surface. It is very well possible that the cross-β structure content of said protein is not changed at all or hardly not changed, but that some protein with cross-β structures has attached to said surface. This is potentially very dangerous because said attached protein comprising cross-β structures can subsequently induce cross-β structures in other proteins or can be thrombogenic/immunogenic/induce inflammatory response when exposed to for example blood or cells. Hence, the invention also provides a method wherein the determining step involves colouring or visualizing with a fluorescent/luminescent compound of said surface with a labelled cross-β structure binding compound. Suitable labels are a fluorescent label, a radioactive label or a peroxidase-conjugated enzyme label. Suitable cross-β structure binding compounds are disclosed in Tables 1-3.

The amount of different treatments, conditions, compounds and/or materials that can be tested in a method according to the invention is enormous. One can for example test one particular treatment (for example storage temperature) or a combination of different treatments (for example storage temperature and the pH of the storage buffer). In one of the embodiments said treatment comprises a physical or mechanical treatment and in another embodiment said treatment comprises a biochemical or chemical treatment. It is also possibly to combine these treatments and hence to subject a protein to a physical or mechanical treatment as well as to a biochemical or chemical treatment, so that the combined effect of these treatments can be assessed in respect of the cross-β structure content of a protein.

Examples of physical or mechanical treatments comprises freezing or thawing or lyophilization of said protein or subjecting said protein to cold or heat or radiation such as X-rays, UV, IR, or subjecting said protein to pressure or air or any combination thereof. Especially a method to determine the effect of freezing or thawing or lyophilization on the cross-β structure content of a protein is important. Enzyme preparations, pharmaceutical compositions and antibody preparations are often frozen and subsequently thawed or lyophilized and reconstituted/re-dissolved. The invention now provides a method to for example test different freezing conditions (slow versus fast or the testing of different solutions in which preparations are frozen) or lyophilization conditions. The treatment that induces the least cross-β structure conformation in a sample (optionally compared to a reference sample) is then selected to treat larger samples which can, in case of for example an enzyme, result in enzyme preparations with better conserved activity. Some other, non-limiting examples of physical/mechanical treatments of a protein are vortexing, sonication, stirring, swirling or shaking.

Examples of biochemical or chemical treatment comprises subjecting a protein to water or high pH or low pH or to a buffer solution or to a liquid comprising a protein or to a liquid medium or to ion strength or to osmosis or to an organic or inorganic detergent or to a radical or contacting a protein with a solid surface, or any combination thereof.

An example of yet another treatment is subjecting a protein to aging. Protein solutions or for example lyophilized proteins are typically stored for long periods. Also blood obtained from volunteers is typically stored for longer periods. It is important that the quality of the blood is kept as high as possible, i.e. the cross-β structure content must be as low as possible. It is assumed that cross-β structure comprising proteins are capable of inducing cross-β structure conformation in the native form of the proteins or in other proteins. If a freshly obtained batch of blood does already comprise some cross-β structure comprising proteins the passing of time (aging) will result in an increase in the cross-β structure content in said batch of blood which eventually decreases the quality of said blood for transfusion. To decrease the effect of aging such a batch of blood is preferably treated with a method to remove compounds with cross-β structure conformation from said blood. Moreover also the storage conditions play an important role in the quality of blood. Important conditions are the type of storage device, the storage temperature, mechanical treatments, the amount of light and so on. The method of the invention provides a fast and convenient method for determining the effect of all these conditions on the cross-β structure content of a protein and hence a method of the invention provides a manner with which the quality of said blood (used for transfusions) is determined. Instead of focusing primarily on the content of amyloidogenic prion protein, our methods focus on cross-β structure conformation in any protein. Moreover, blood comprising protein with cross-β structures that is subsequently used for blood transfusion can results in immunogenic and/or thrombogenic and/or inflammatory responses in the receiving mammal (for example a human). Such responses can now be at least partly prevented or at least partly decreased and more preferably completely prevented by checking all the steps involved in obtaining the blood, storing the blood and providing the blood to a patient in need thereof for their cross-β structure inducing capability and selecting conditions that preferably prevent cross-β structure formation.

The successful application of solid surfaces like for example the application of solid surfaces in heart valves, heart aid devices (pacemaker), heart pumps, haemodialysis membranes, (closed loop) insulin delivery system, artificial implant applications, medical devices, equipment during heart surgery, extracorporeal device, cardiopulmonary bypass devices, prosthetic devices, bone implants, artificial organs, vascular grafts, vascular prostheses, stents, depend largely on their biocompatibility. Such devices are for example prepared from carbons, glass, ceramics polymers, hydrogels, collagen, polyurethanes, negatively charged polyamide, polysulfone, polystyrene, stainless steel, (carbon-coated) polytetrafluoroethylene, titanium, aluminium, iridium, indium, nickel, tantalum, tin, zirconium, Dacron, and presently, heparin or albumin-heparin conjugate is widely used as a clinical anticoagulant on such devices.

The invention now provides a method to test (existing or newly designed/produced) solid surfaces for their biocompatibility. Preferably said solid surface is a metal or plastic or wooden or glass or biochemical compound, like for example cellulose, liposomes, carbohydrates, or chemical compounds, like for example dendrimers, carbon, polymers, surface or any combination thereof. Examples of currently used metals are titanium, aluminium, iridium, indium, tantalum, tin, titanium or zirconium. These metals are used today in implants as well as in blood-contacting devices. Their biocompatibility is now tested more easily by performing a method according to the invention and selecting a metal or a metal alloy that essentially does not increase the amount of cross-β structure conformation.

In one preferred embodiment, the invention provides a method for selecting a biocompatible material that essentially preserves the cross-β structure content of a protein comprising

determining in a reference sample the cross-β structure content of said protein

contacting said protein with a biocompatible surface that is expected to have an effect on the cross-β structure content to obtain a test sample

determining in said test sample the cross-β structure content of said protein

selecting the biocompatible material that essentially preserves the cross-β structure content of said protein, i.e. selecting the material that does not increase the cross-β structure content of a protein preferably a protein solution (for example blood).

Presently new biocompatible materials are, amongst others, designed and prepared by coating a biocompatible material (for example with heparin or an albumin/heparin conjugate). Such a coated biocompatible material is with a method according to the invention also easily checked for its effect on the cross-β structure content of a protein. Examples of used coatings are proteins or fragments thereof.

Upon performing a method as described above a suitable (coated) biocompatible material is selected. A suitable/selected (coated) biocompatible material obtainable by a method according to the invention is also claimed herein.

Based on our findings, suitable coated biomaterials are now designed. A coating suitable for a biocompatible material based on proteins or fragments thereof that are more or less resistant to cross-β structure formation are very useful. Examples are non-amyloid human fibrin peptide NH₂-KRLEVDIDIK-COOH FP10 (ref. ³), the murine islet amyloid polypeptide decapeptide fragment NH₂-SNNLGPVLPP-COOH Δ murine IAPP (ref ¹³) and even single amino acids. Because these coatings cannot (or hardly not) assume a cross-β structure conformation these coating are also not (or hardly not) capable of inducing cross-structures in contacting proteins.

A suitable/selected (coated) biocompatible material obtainable by a method according to the invention or a biocompatible material designed on the above described findings is preferably used for preparing a biocompatible part/device/material/product.

Non-limiting examples of a biocompatible part/device/material/product is a stent, heart valves, heart aid devices (pacemaker), heart pumps, haemodialysis membranes, (closed loop) insulin delivery system, vascular grafts, artificial implant applications, medical devices, equipment during heart devices, extracorporeal (circulation) device, cardiopulmonary bypass devices, prosthetic devices, bone implants, artificial organs or vascular prostheses

In another preferred embodiment, the invention provides a method for selecting a material suitable for the interior of a bioreactor comprising

determining in a reference sample the cross-β structure content of a protein

contacting said protein with a material suitable for the interior of a bioreactor that is expected to have an effect on the cross-β structure content to obtain a test sample

determining in said test sample the cross-β structure content of said protein

selecting the material suitable for the interior of a bioreactor that essentially preserves the cross-β structure content of said protein, i.e. selecting the material that does not increase the cross-β structure content of a protein preferably a protein solution.

A bioreactor as used herein embraces a large-scale bioreactor as well as a smaller bioreactor such as an Eppendorf tube or a well of for example an ELISA plate.

By selecting a material to be used for the interior of a bioreactor with a method according to the invention, material is selected that does not increase the cross-β structure content of a protein (solution). For a large-scale bioreactor, for example large-scale production of a micro-organism that produces a secreted protein, this has the effect that the produced, secreted protein will not adopt a cross-β structure conformation or not as much compared to another material. As a result, the produced protein is of higher quality because it comprises a lower cross-β structure content. For a small-scale bioreactor, for example an Eppendorf tube or an incubation well of an ELISA plate, this has the effect that a for example performed enzymatic assay is not or hardly not or considerably less compared to other materials disturbed by the presence of cross-β structure conformation inducing compounds and hence that a better view (more relevant data) is obtained in respect of the performed assay and that artefacts induced by the used material can be as much as possible avoided.

The invention also provides a material suitable for the interior of a bioreactor obtainable by a method according to the invention. Such a bioreactor is especially useful for preparing a bioreactor.

In yet another embodiment, the invention provides a method for selecting a material suitable for the interior of a storage device that essentially preserves the cross-β structure content of a protein comprising

determining in a reference sample the cross-β structure content of said protein

subjecting said protein to a material suitable for the interior of a storage device that is expected to have an effect on the cross-β structure content to obtain a test sample

determining in said test sample the cross-β structure content of said protein

selecting the material suitable for the interior of a storage device that essentially preserves the cross-β structure content of said protein.

It is to be understood that the term “preserves the cross-β structure content of a protein” also includes the situation in which the reference sample does not (or hardly not) comprise any cross-β structure and this is maintained during the treatment.

In a preferred embodiment, the subjecting step comprises contacting said protein with a material suitable for the interior of a storage device.

Proteins or protein solutions are often stored for longer periods in storage devices. It is important that the material of said storage devices does not induce cross-β structure conformation formation in said protein or protein solution. Application of a method according to the invention results in material suitable for the interior of a storage device that is subsequently used for preparing a storage device.

Examples of useful applications of a method according to the invention are provided above and even more examples are provided below. In general it can be said that if one wants to study or obtain a protein with a particular property, it is important to check (if possible) each and every treatment on their cross-β structure inducing capabilities on said protein. If for example a protein is used in the food industry it is important to check the production, purification and storage conditions. If one wants to study the activity of a protein (for example an enzyme) it is important to study all the conditions to which such a protein is subjected.

Other, non-limiting, applications of a method according to the invention are

testing of conditions for growing crystals for protein crystallography purposes; some of the presently used conditions result in the formation of cross-β structure conformation in a protein and hence hampers the growth of high-quality crystals of said protein; conditions (to be) used in crystallography are now tested for their cross-β structure inducing capability and a selection is made for conditions that do not or hardly not induce the formation of cross-β structure conformation in a protein;

testing of materials used in protein purifications; independent of the source of protein (naturally expressed or recombinantly expressed) proteins are typically subjected to one or multiple purification steps to obtain high grade (pharmaceutical) preparations. All material used in such purifications, such as column material, dialysis membranes, membranes used for concentration, is checked with a method according to the invention and materials are selected that do not or hardly not induce cross-β structure conformation formation in the to be purified protein;

testing of conditions for protein refolding from an aggregated state to a native fold; independent of the source of the protein with non-native fold (naturally expressed or recombinantly expressed; for example Escherichia coli inclusion bodies), proteins are typically subjected to exposure to one or more solutions that putatively aid the folding from a non-native fold to a native fold. The solutions are now checked with a method according to the invention for their propensity to induce the cross-β structure conformation in proteins by testing the content of cross-β structure conformation in the proteins after the exposure to the solutions. Solutions can now be selected that do not result in cross-β structure conformation and thus may aid the adoption of a native fold.

selection and development of cell culture disposables or laboratory equipment in general

Sometimes it is not possible to avoid that a certain cross-β structure content in a protein is formed. If a pharmaceutical composition comprising a protein is delivered to a mammal (non-human or human) via a syringe/injection needle, the protein present in said pharmaceutical composition is typically exposed to a relative high shear stress which perhaps induces cross-β structure conformation in said protein. The cross-β structure conformation formation can at least partly be reduced by testing the material used for the needle and by adjusting the pore size of the needle and by adjusting the flow through the needle.

It is clear that if a certain cross-β structure inducing treatment cannot be avoided it is possible to remove induced cross-β structures in a protein. This is explained in more detail in one of our co-pending applications.

In yet another embodiment, the invention provides a kit comprising all the essential means for detecting a cross-β structure in a protein. Examples of such means are a solid surface for immobilization (for example beads or an ELISA plate), a (labelled) compound of Table 1 or 2 or 3, means for visualization, positive and/or negative controls. Such a kit is for example suitable for an enzymatic assay, such as the tPA/plasminogen enzymatic assay or the FXII enzymatic assay or the FXII/prekallikrein/high molecular weight kininogen enzymatic assay or an enzymatic assay based on the use of HGFA.

The invention will be explained in more detail in the following examples, which is not limiting the invention.

TABLE 1
cross-β structure binding compounds

| | | |
|---|---|---|
| Congo red | Chrysamine G | Thioflavin T |
| 2-(4′-(methylamino)phenyl)-6- | Any other | Glycosaminoglycans |
| methylbenzothiaziole | amyloid-binding | |
| | dye/chemical | |
| Thioflavin S | Styryl dyes | BTA-1 |
| Poly(thiophene acetic acid) | conjugated | Ellagic acid |
| | polyeclectrolyte | |
| | PTAA-Li | |

TABLE 2
Proteins that bind to and/or interact with misfolded proteins and/or with
proteins comprising cross-β structure

| | | |
|---|---|---|
| Tissue-type plasminogen | Finger domain(s) of tPA, factor | Apolipoprotein E |
| activator | XII, fibronectin, HGFA | |
| Factor XII | Plasmin(ogen) | Matrix metalloprotease-1 |
| Fibronectin | 75 kD-neurotrophin receptor | Matrix metalloprotease-2 |
| | (p75NTR) | |
| Hepatocyte growth factor | α2-macroglobulin | Matrix metalloprotease-3 |
| activator | | |
| Serum amyloid P component | High molecular weight kininogen | Monoclonal antibody |
| | | 2C11(F8A6)^{‡} |
| C1q | Cathepsin K | Monoclonal antibody 4A6(A7)^{‡} |
| CD36 | Matrix metalloprotease 9 | Monoclonal antibody 2E2(B3)^{‡} |
| Receptor for advanced glycation | Haem oxygenase-1 | Monoclonal antibody 7H1(C6)^{‡} |
| endproducts | | |
| Scavenger receptor-A | low-density lipoprotein receptor- | Monoclonal antibody 7H2(H2)^{‡} |
| | related protein (LRP, CD91) | |
| Scavenger receptor-B | DnaK | Monoclonal antibody 7H9(B9)^{‡} |
| ER chaperone Erp57 | GroEL | Monoclonal antibody 8F2(G7)^{‡} |
| Calreticulin | VEGF165 | Monoclonal antibody 4F4^{‡} |
| Monoclonal conformational | Monoclonal conformational | Amyloid oligomer specific |
| antibody WO1 (ref. (O'Nuallain | antibody WO2 (ref. (O'Nuallain | antibody (ref. (Kayed et al., |
| and Wetzel, 2002)) | and Wetzel, 2002)) | 2003)) |
| formyl peptide receptor-like 1 | α(6)β(1)-integrin | CD47 |
| Rabbit anti-albumin-AGE | CD40 | apo A-I belonging to small high- |
| antibody, Aβ-purified^{a)} | | density lipoproteins |
| apoJ/clusterin | 10 times molar excess PPACK, 10 mM | CD40-ligand |
| | εACA, (100 pM-500 nM) | |
| | tPA²⁾ | |
| macrophage scavenger receptor | broad spectrum (human) | BiP/grp78 |
| CD163 | immunoglobulin G (IgG) | |
| | antibodies (IgIV, IVIg) | |
| Erdj3 | haptoglobin | |

^{‡}Monoclonal antibodies developed in collaboration with the ABC-Hybridoma Facility, Utrecht University, Utrecht, The Netherlands.
^{a)}Antigen albumin-AGE and ligand Aβ were send in to Davids Biotechnologie (Regensburg, Germany); a rabbit was immunized with albumin-AGE, antibodies against a structural epitope were affinity purified using a column with immobilized Aβ.
²⁾PPACK is Phe-Pro-Arg-chloromethylketone (SEQ-ID 8), εACA is ε-amino caproic acid, tPA is tissue-type plasminogen activator

TABLE 3
Proteins involved in the “Cross-beta structure pathway

| | | |
|---|---|---|
| Monoclonal antibody 4B5 | Heat shock protein 27 | Heat shock protein 40 |
| Monoclonal antibody 3H7^{‡} | Nod2 (= CARD15) | Heat shock protein 70 |
| FEEL-1 | Pentraxin-3 | HDT1 |
| LOX-1 | Serum amyloid A proteins | GroES |
| MD2 | Stabilin-1 | Heat shock protein 90 |
| FEEL-2 | Stabilin-2 | CD36 and LIMPII analogous-I |
| | | (CLA-1) |
| Low Density Lipoprotein | LPS binding protein | CD14 |
| C reactive protein | CD45 | Orosomucoid |
| Integrins | alpha-1 antitrypsin | apo A-IV-Transthyretin complex |
| Albumin | Alpha-1 acid glycoprotein | β2-glycoprotein I |
| Lysozyme | Lactoferrin | Megalin |
| Tamm-Horsfall protein | Apolipoprotein E3 | Apolipoprotein E4 |
| Toll-like receptors | Complement receptor CD11b/CD18 | CD11d/CD18 (subunit aD) |
| | (Mac-1, CR3) | |
| CD11b2 | CD11a/CD18 (LFA-1, subunit aL) | CD11c/CD18 (CR4, subunit aX) |
| Von Willebrand factor | Myosin | Agrin |
| Perlecan | Chaperone60 | b2 integrin subunit |
| proteins that act in the unfolded | proteins that act in the endoplasmic | Macrophage receptor with |
| protein response (UPR) pathway | reticulum stress response (ESR) | collagenous structure (MARCO) |
| of the endoplasmic reticulum | pathway of prokaryotic and | |
| (ER) of prokaryotic and | eukaryotic cells | |
| eukaryotic cells | | |
| 20S | CHAPERONE16 family members | HSC73 |
| HSC70 | translocation channel protein Sec61p | 26S proteasome |
| 19S cap of the proteasome | UDP-glucose:glycoprotein glucosyl | carboxy-terminus of |
| (PA700) | transferase (UGGT) | CHAPERONE70-interacting |
| | | protein (CHIP) |
| Pattern Recognition Receptors | Derlin-1 | Calnexin |
| Bcl-2 asociated athanogene | GRP94 | Endoplasmic reticulum p72 |
| (Bag-1) | | |
| (broad spectrum) (human) | proteins that act in the endoplasmic | The (very) low density lipoprotein |
| immunoglobulin M (IgM) | reticulum associated degradation | receptor family |
| antibodies | system (ERAD) | |
| Fc receptor | | |

^{‡}Monoclonal antibodies developed in collaboration with the ABC-Hybridoma Facility, Utrecht University, Utrecht, The Netherlands.

### EXAMPLES

### Materials and Methods Example 1

Preparation of Amyloid-Like Aggregates of γ-Globulins

Amyloid preparations of human γ-globulins were made as follows. Lyophilized γ-globulins (G4386, Sigma-Aldrich, Zwijndrecht, The Netherlands) were dissolved in a 1(:)1 volume ratio of 1,1,1,3,3,3-hexafluoro-2-propanol and trifluoroacetic acid and subsequently dried under an air stream. Dried γ-globulins were dissolved in H₂O to a final concentration of 1 mg ml⁻¹ and kept at room temperature for at least three days. Aliquots were stored at −20° C. The presence of cross-β structure conformation was established by enhanced ThT fluorescence, enhanced Congo red fluorescence, tPA binding, tPA-mediated plasminogen (Plg) activation and appearance as string-like aggregates on transmission electron microscopy (TEM) images (see below).

### Preparation of Cross-β Structure Conformation Rich Advanced Glycation Endproducts (RAGE)

Human haemoglobin (Hb, Sigma-Aldrich, H7379, Zwijndrecht, The Netherlands) at 5 mg ml⁻¹ was incubated for 32 weeks at 37° C. with PBS containing 1 M of glucose-6-phosphate (g6p) and 0.05% m/v of NaN₃. In control solutions, g6p was omitted. After incubations, solutions were extensively dialyzed against distilled H₂O and, subsequently, stored at 4° C. Protein concentrations were determined with Advanced protein-assay reagent ADV01 (Cytoskeleton, Denver, Colo., USA). Glycation and formation of AGE was confirmed by measuring intrinsic fluorescent signals from AGE; excitation wavelength 380 nm, emission wavelength 435 nm. In addition, binding of AGE-specific antibodies was determined. Presence of cross-β structure conformation in Hb-AGE was conformed by tPA binding, tPA activation, FXII activation, circular dichroism spectropolarimetry analyzes, transmission electron microscopy imaging of fibrillar structures and by Congo red fluorescence measurements.

Preparation of Peptide Samples with Cross-β Structure Conformation and of Controls without Amyloid-Like Structure

Peptide batches were prepared as follows. Human Aβ(1-40) Dutch type (DAEFRHDSGYEVHHQKLVFFAQDVGSNKGAIIGLMVGGVV) was disaggregated in a 1:1 (v/v) mixture of 1,1,1,3,3,3-hexafluoro-2-isopropyl alcohol and trifluoroacetic acid, air-dried and dissolved in H₂O at 1 or 10 mg ml⁻¹. After three days at 37° C., solutions were kept at room temperature for two weeks, before storage at 4° C. Non-amyloid fragment FP10 of human fibrin α-chain(148-157) (KRLEVDIDIK)^{3,16} was dissolved at a concentration of 1 mg ml⁻¹ in H₂O and stored at 4° C. Peptide solutions were tested for the presence of amyloid conformation by ThT or Congo red fluorescence as described^{1,17,18}. ThT- and Congo red fluorescence was enhanced for amyloid Aβ, and not for non-amyloid FP10 or freshly dissolved Aβ.

### Plasminogen-Activation Assay and Factor XII-Activation Assay

Plasmin (Pls) activity was assayed as described³. Peptides and proteins that were tested for their stimulatory ability were regularly used at 100 μg ml⁻¹. The tPA (Actilyse, Boehringer-Ingelheim, Alkmaar, The Netherlands) and Plg (purified from human outdated plasma by lysine affinity chromatography) concentrations were 200 pM and 1.1 μM, respectively, unless stated differently. Chromogenic substrate S-2251 (Chromogenix, Instrumentation Laboratory SpA, Milano, Italy) was used to measure Pls activity. Conversion of zymogen FXII (#233490, Calbiochem, EMD Biosciences, Inc., San Diego, Calif.) to proteolytically active FXII (FXIIa) was assayed by measurement of the conversion of chromogenic substrate Chromozym-PK (Roche Diagnostics, Almere, The Netherlands) by kallikrein. Chromozym-PK was used at a concentration of 0.3 mM. FXII, human plasma prekallikrein (#529583, Calbiochem) and human plasma cofactor high-molecular weight kininogen (#422686, Calbiochem) were used at concentrations of 1 μg ml⁻¹. The assay buffer contained HBS (10 mM HEPES, 4 mM KCl, 137 mM NaCl, 5 μM ZnCl₂, pH 7.2). Assays were performed using microtiter plates (catalogue number 2595, Costar, Cambridge, Mass., USA). Peptides and proteins were tested for their ability to activate FXII. 150 μg ml⁻¹ kaolin, an established activator of FXII was used as positive control and solvent (H₂O) as negative control. The conversion of Chromozym-PK was recorded kinetically at 37° C. for at least 60 minutes. Assays were done in duplicates. In control wells FXII was omitted from the assay solutions and no conversion of Chromozym-PK was detected.

Thioflavin T fluorescence

Fluorescence of ThT-amyloid-like protein/peptide adducts was measured as follows. Solutions of 25 μg ml⁻¹ of protein or peptide preparations were prepared in 50 mM glycine buffer pH 9.0 with 25 μM ThT. Fluorescence was measured at 485 nm upon excitation at 435 nm. Background signals from buffer, buffer with ThT and protein/peptide solution without ThT were subtracted from corresponding measurements with protein solution incubated with ThT. Regularly, fluorescence of amyloid-β was used as a positive control, and fluorescence of FP10, a non-amyloid fibrin fragment³, and buffer was used as a negative control. Fluorescence was measured in triplicate on a Hitachi F-4500 fluorescence spectrophotometer (Hitachi, Ltd., Tokyo, Japan).

### Transmission Electron Microscopy (TEM) Imaging

For TEM analysis of protein en peptide solutions grids were prepared according to established procedures. Samples were applied to 100-mesh copper grids with carbon coated Formvar (Merck, Germany), and subsequently washed with PBS and H₂O. Grids were applied to droplets of 2% (m/v) methylcellulose with 0.4% (m/v) uranyl acetate pH 4. After a 2′-minutes incubation grids were dried on a filter. Micrographs were recorded at 80 kV, at suitable magnifications on a JEM-1200EX electron microscope (JEOL, Japan).

Analysis of Protein Structure after Exposure to Dextran Sulphate, Kaolin and CpG-ODN Surfaces

Lyophilized proteins were dissolved in HEPES-buffered saline (HBS, 10 mM HEPES, 4 mM KCl, 137 mM NaCl, pH 7.2) to a final concentration of 2 mg ml⁻¹. Proteins were gently dissolved on a roller at room temperature for 10 min, at 37° C. for 10 min and again at room temperature for 10 min. Kaolin (6564, Genfarma, Zaandam, The Netherlands) suspension and dextran sulphate Mw 500,000 Da (DXS500k, Pharmacia, Amersham Biosciences Europe, Roosendaal, The Netherlands) stock solutions of 500 μg ml⁻¹ were prepared in HBS. Bovine serum albumin (BSA, ICN, #160069, fraction V, Irvine, Calif., USA), lysozyme (ICN, 100831), γ-globulins, endostatin, a recombinantly produced fragment of human collagen XVIII fragment (EntreMed, Inc., Rockville, Md.) and FXII (Calbiochem, 233490) were diluted 1:1 in HBS alone or in HBS with kaolin or DXS500k. Human pooled citrated plasma was diluted 40× in HBS before use to obtain an estimated total protein concentration of 2 mg ml⁻¹, and subsequently diluted 1:1 in buffer or surface solution/suspension. Control protein samples and the protein samples with adjuvant were incubated overnight at 4° C. on a roller.

After incubation, 25 μL of the samples were analyzed for ThT binding (see above). Fluorescence of the buffer or the surfaces was recorded for background subtraction purposes. Amyloid-β(1-40) E22Q was used as a positive control. Alternatively, control proteins and proteins incubated with DXS500k were immobilized on Greiner Microlon high-binding ELISA plates (Greiner Bio-One GmbH, Frickenhausen, Germany). Wells were blocked with Blocking Reagent (catalogue number 11112589001, Roche Diagnostics, Almere, The Netherlands). Glycated haemoglobin (Hb-AGE) was immobilized as a positive control for tPA binding to a protein aggregate with amyloid-like properties. Hb-AGE i) appears as fibrous structures under the transmission electron microscope (not shown), ii) contains an increased amount of β-sheet secondary structure, as determined with circular dichroism spectropolarimetry (not shown), and iii) enhances Congo red fluorescence (not shown). Subsequently the wells were incubated with concentration series of tPA (Actilyse, Boehringer-Ingelheim, Alkmaar, The Netherlands) or a truncated form of tPA (K2P-tPA, Rapilysin, Boehringer-Ingelheim, Alkmaar, The Netherlands) lacking three amino-terminal domains including the fibronectin type I (F) domain, in the presence of 10 mM εACA. Binding of tPA and K2P-tPA was assessed with monoclonal antibody 374b (American Diagnostica, Tebu-Bio, The Netherlands), peroxidase-conjugated rabbit anti-mouse immunoglobulins (RAMPO, P0260, DAKOCytomation, Glostrup, Denmark) and stained with 3′3′5′5′-tetramethylbenzidine (TMB, catalogue number 4501103, buffer, catalogue number 4501401, Biosource Int., Camarillo, Calif., USA). In addition, lysozyme was incubated with 250 μg ml⁻¹ DXS500k and TEM images are recorded with lysozyme with DXS500k and with DXS500k alone. To determine the ThT-fluorescence inducing capacity of multimeric molecules, CpG-ODN (Coley Pharmaceutical Group, MA, USA) at 21.4 μg ml⁻¹ was mixed with 1 mg ml⁻¹ of chicken egg-white lysozyme (#62971, Fluka, Sigma-Aldrich), BSA, endostatin, human γ-globulins, human β2-glycoprotein I (β2GPI) purified from plasma as described¹⁹ and recombinant human β2GPI obtained as described²⁰, and incubated o/n on a roller at 4° C., before ThT fluorescence measurements. For this purpose, protein solutions at 2 mg ml⁻¹ were ultracentrifuged for 1 h at 100,000*g before use, and subsequently diluted 1:1 in buffer with 42.9 μg ml⁻¹ CpG-ODN. Also TEM images are taken with the CpG-ODN only, CpG-ODN with lysozyme and lysozyme only samples.

### Influence of Plastic Surfaces on Protein Conformation

To analyze the influence of 96-well plate material on tPA activation in aqueous buffer, we performed activation assays in wells of four different plates. We included 8-strip wells of an Immobilizer Amino plate (Nunc, Roskilde, Denmark), Peptide Immobilizer plate (Exiqon, Vedbaek, Denmark), high-binding ELISA plate (Costar, catalogue number 9102, Corning, N.Y., USA) and a hydrophobic ELISA plate (Costar catalogue number 2595, Corning, N.Y., USA). The Immobilizer plates are made of polystyrene. Immobilizer plates contain organic spacers that expose a reactive group that will covalently bind —NH₂, —SH and —OH groups in polypeptides. The reactive groups can be blocked by Tween-20. The Costar 2595 plate is made of vinyl, the Costar 9102 plate is made of polystyrene, that is γ-irradiated for tissue culture purpose. Wells were used directly in de assay or the wells of the Immobilizer plates were blocked with PBS containing 1% v/v Tween-20 and wells of the Costar plates were blocked with Blocking Reagent (catalogue number 11112589001, Roche Diagnostics, Almere, The Netherlands). Blocked and unblocked wells were washed twice with H₂O before use. In one half of the experiments, a cofactor for tPA-mediated Pls formation was omitted. In the second half of the experiments, 5 μg ml⁻¹ amyloid γ-globulins were included as cofactor BSA, ovalbumin (OVA) and haemoglobin (Hb), using a tPA ELISA. Next to an Immobilizer Amino plate (Nune, Roskilde, Denmark), a γ-irradiated cell-culture grade negatively charged ELISA plate (Costar, catalogue number 9102, Corning, N.Y., USA) and a hydrophobic vinyl ELISA plate (Costar catalogue number 2595, Corning, N.Y., USA), a polystyrene high-binding Microlon (Greiner Bio-One GmbH, Frickenhausen, Germany) was included in the test. BSA, OVA (A-7641, Sigma-Aldrich, Zwijndrecht, The Netherlands) and Hb (Hb, H-7379, Sigma-Aldrich) were coated at 5 μg ml⁻¹ in 50 mM carbonate buffer pH 9.6 on the Nunc, Greiner and Costar 2595 plates. In control wells only coat buffer was coated. Plates are blocked with Blocking reagent (catalogue number 11112589001, Roche Diagnostics, Almere, The Netherlands) (Costar, Greiner) or with 1% Tween-20 in PBS (Nunc).

Concentration series of tPA in the presence of 10 mM ε-amino caproic acid (εACA) is added to the wells and binding of tPA is assessed with monoclonal antibody 374b (American Diagnostica, Tebu-Bio, The Netherlands), peroxidase-conjugated rabbit anti-mouse immunoglobulins (RAMPO, P0260, DAKOCytomation, Glostrup, Denmark) and stained with 3′3′5′5′-tetramethylbenzidine (TMB, catalogue number 4501103, buffer, catalogue number 4501401, Biosource, Camarillo, Calif., USA). Coat efficiency was established with rabbit polyclonal anti-BSA antibody A-0001 (DAKOCytomation, Glostrup, Denmark), monoclonal mouse ascites anti-OVA antibody A-6075 (Sigma-Aldrich, Zwijndrecht, The Netherlands) and rabbit polyclonal anti-Hb antibody A-0118 (DAKOCytomation). Signals obtained with these antibodies were used for scaling of the signals obtained with tPA-374b on the different plates.

The Costar 9102 plate was used for a slightly different approach. OVA, Hb, BSA and tPA, all at 5 μg ml⁻¹ except tPA (6 μg ml⁻¹), were immobilized in 50 mM carbonate buffer pH 9.6. Next, the plate was blocked with Blocking Reagent (Roche) containing 1% m/v proteolytically degraded purified gelatin. Coating of the proteins was assessed with protein specific antibodies. For comparison, wells were first blocked and then incubated with the protein solutions in the carbonate coat buffer. In this way, the block efficiency will become clear. To address the possibility that the plate induces tPA binding sites in the Blocking Reagent, blocked wells are incubated for 1 h at room temperature with swirling, with concentration series of tPA in the presence of 10 mM ε-amino-caproic-acid (εACA), to avoid binding of the kringle2 domain of tPA to lysine- and arginine residues, and with OVA, BSA and Hb. Binding buffer is phosphate buffered saline (PBS, 140 mM sodium chloride, 2.7 mM potassium chloride, 10 mM disodium hydrogen phosphate, 1.8 mM potassium dihydrogen phosphate, pH 7.3) with 0.1% v/v Tween-20. Putative binding of the proteins is assessed with the protein specific antibodies listed above. The tPA concentration series is also applied to similarly blocked wells of the Nunc Immobilizer Amino-, the Costar 2595- and the Greiner Microlon high-binding ELISA plates, for comparison. Concentration series were 0/3/9/27/81 nM for tPA, 0/9.3/18.6/37.3/74.5 nM for BSA, 0/14.5/29/58/116 nM for OVA and 9.7/19.4/36.8/73.5 nM for Hb. Molecular weights that were used to calculate molar concentrations are 70 kDa for tPA, 67 kDa for BSA, 43 kDa for OVA and 68 kDa for Hb.

In another experiment lysozyme (2 mg ml⁻¹) in HBS was exposed to a Microlance-3 needle (Beckton-Dickinson Labware, catalogue number 301750, 19G2″, Droheda, Ireland) for 72 h at 37° C. Controls were lysozyme not exposed to the needle, buffer and needle, separately. Change in protein structure was monitored with 1:1 diluted solutions in HBS in a Plg-activation assay using 400 pM tPA, 20 μg ml⁻¹ Plg, 0.5 mM Pls substrate S-2251 (Chromogenix, Instrumentation Laboratory SpA, Milan, Italy) in a 96-wells plate (Costar, catalogue number 2595, Corning, N.Y., USA). As a positive control for the Plg-activation assay 5 μg ml⁻¹ amyloid γ-globulins were used Buffer only was used as a negative control. The signal of the negative control was subtracted from all other signals. Data points at t=0 were used to scale all data sets. In addition to lysozyme, 2 mg ml⁻¹ γ-globulins were exposed to the Microlance-3 needle for 72 h at 4° C. ThT fluorescence measurements were performed with these samples, and 25 μg ml⁻¹ Aβ (positive control) and non-amyloid fibrin peptide FP10 (negative control).

### Results Example 1

### Influence of Kaolin and DXS500k on Protein Stability

To test the influence of negatively charged polymer DXS500k and of particles of the mineral kaolin on the generation of cross-β structure, BSA, γ-globulins, lysozyme, FXII, endostatin and diluted plasma were exposed to kaolin or DXS500k, two compounds that are well known for their ability to activate FXII but are also used as adjuvant²¹⁻²⁴. Subsequently, ThT fluorescence was determined. FXII was only exposed to DXS500k. After subtraction of background signals, kaolin induces an increased ThT fluorescence signal of 1.6 up to 6.6 fold. DXS500k enhances ThT fluorescence 2.6 times (FXII) to 17.8 times (BSA) (FIG. 1A). In an ELISA binding of tPA and K2P-tPA (a truncated tPA lacking the cross-β structure binding fibronectin type I domain) to immobilized control proteins and mixtures of proteins with DXS500k was assessed (FIG. 1A). K2P-tPA did not bind to any of the proteins or DXS500k-protein mixtures (not shown). Exposure of proteins or diluted plasma to DXS500k increased tPA binding with a factor 1.3 (BSA) up to 10.5 (endostatin), when compared to the binding of tPA to proteins that were incubated with buffer only. The ThT fluorescence data and the tPA binding data show that exposure of proteins to mineral kaolin particles and DXS500k polymers induces or enhances amyloid-like properties in proteins.

Next, we tested whether the amyloid-like structures that are introduced in proteins upon exposure to kaolin or DXS500k influences FXII activation. For this purpose we used assay conditions during which FXII is not or hardly activated by kaolin or DXS500k (FIG. 1B-E). Under these conditions FXII can be activated by adding BSA (FIG. 1B, D) and endostatin (FIG. 1C, E). Neither BSA or endostatin alone, nor kaolin or DXS500k alone are efficient activators of FXII, whereas combinations of surface and protein cofactor results in FXII and subsequent prekallikrein activity. Taken together, BSA or endostatin that is denatured by surfaces of DXS500k or kaolin act as efficient activator of FXII. These data further show that exposure of proteins to the polymer surface of DXS500k or to the clay particle surface of kaolin results in refolding of the native protein into an amyloid-like structure with cross-β structure conformation.

Further evidence for an influence of DXS500k on protein stability comes from analysis of TEM images. This was recorded with a lysozyme solution, either exposed to DXS500k or without DXS500k (FIG. 1F-H). A few aggregates were found in the lysozyme solution that was not exposed to DXS500k (FIG. 1F). A large amount of large networks of strings of globular aggregates were observed when lysozyme was exposed to DXS500k (FIG. 1H). The needles in the DXS500k solution (FIG. 1G) disappeared after exposure to lysozyme. When 10.4 or 21.7 μg ml⁻¹ CpG-ODN was incubated with 1 mg ml⁻¹ lysozyme or endostatin for 30 min. at room temperature, an increase in ThT fluorescence of approximately 8 to 7 times for lysozyme and 39 to 56 times for endostatin was observed, respectively (FIG. 1I, J)). In addition, overnight exposure at 4° C. of 1 mg ml⁻¹ BSA, endostatin, plasma β2GPI or rec. β2GPI to 21.4 μg ml⁻¹ CpG-ODN resulted in increased ThT fluorescence with approximately a factor 3, 10, 2 and 5, respectively (FIG. 1K). With these assay conditions no effect was seen with lysozyme and γ-globulins. Analysis with TEM of CpG-ODN, lysozyme and lysozyme with CpG-ODN, all after overnight incubation, revealed that small needles were present in the CpG-ODN solution (FIG. 1L) and that few aggregates were present in the lysozyme solution (FIG. 1F). When CpG-ODN and lysozyme were incubated together, a high density of relatively thick aggregates were observed that seem to be composed of strings of globular precipitates (FIG. 1M). These data altogether show that structural changes accompanied with the formation of aggregates with amyloid-like properties, occurs upon exposure of the tested proteins to CpG-ODN.

### Influence of Plastic Surfaces on Protein Structure

To test the hypothesis that surfaces, including ELISA plates and biomaterials induce cross-β structure we tested four different type of plates for their influence on tPA-mediated Plg activation. Immobilizer Amino plates of polystyrene with a coated organic spacer (Nunc, Exiqon), a polystyrene γ-irradiated plate (Costar 9102) and a vinyl plate (Costar 2595). Plg and tPA were mixed with buffer (FIG. 2A, C) or with amyloid γ-globulins (FIG. 2B, D). The influence of blocking the plates prior to the tPA activation was also assessed (FIG. 2A, B vs. C, D). Immobilizer amino plates blocked with 0.1% v/v Tween-20 resulted in some Pls activity even when amyloid γ-globulins was omitted. No activity was observed in unblocked plates without cofactor. Blocking had no influence on the activity in the presence of amyloid γ-globulins. Costar 9102 plates that were unblocked did not result in activation of tPA. Blocking the plate with Blocking reagent (Roche) induced some activity. The unblocked, Costar 2595 plate was unique in inducing some activity in the absence of amyloid γ-globulins and no increase when the plate was blocked. Overall, when amyloid γ-globulins was present blocking had no influence on the final activity. We conclude from these experiments that the combination of polystyrene plates with a coated organic spacer, blocked with Tween-20 has some denaturing activity towards tPA and/or Plg, thereby forming the necessary cross-β structure rich template for tPA-mediated Pls generation. The vinyl Costar 2595 has this denaturing capacity irrespective of a block step. Putatively, this intrinsic denaturing capacity of the vinyl is also at the basis of the relatively high Pls activity when amyloid γ-globulins is added as a cross-β structure template. A small amount of denatured Plg, tPA or γ-globulins at the vinyl surface may facilitate the first step of the reaction by providing a solid surface in which the firstly generated Pls molecules can generate C-terminal Lys/Arg residues which serve as binding sites for Plg that will accelerate further Pls generation. When less denatured protein is immobilized in the other three different plates, the first steps of the reaction has to occur in solution that apparently may slow down the reaction.

In a next series of experiments we analyzed the influence of various types of ELISA plates on the structural stability of a protein that is brought in contact with the plastic surface, and on the varying degree of protein adhesion. First, coat efficiency on the Nunc Immobilizer Amino plate, the Costar 2595 plate and the Greiner Microlon plate were assessed (FIG. 2E-G). BSA binds to all three plates to a similar extent, as detected with an anti-BSA antibody. In contrast, for both OVA and Hb less binding is observed in Costar 2595 plates. Differences in the coated amounts are used as correction factors in the subsequent experiment in which tPA binding to the three proteins coated onto the three different plates was assessed (FIG. 2H-J). Apparently, all ELISA plates introduce tPA binding sites in the coated proteins, or the binding site are already present before coating. However, still differences are seen between the amount of tPA bound to BSA coated to the Nunc plate or to the other plates. The Nunc plate seems to introduce more tPA binding sites, indicative for formation of more cross-β structure conformation. Differences are also seen with Hb (FIG. 2J). Again the Nunc plate induces the strongest tPA binding, a phenomenon that is seen to a lesser extent with OVA (FIG. 2I). These data demonstrate that exposure of a protein to various plastic surfaces can introduce amyloid-like properties in proteins and to a different extent.

This study was expanded with a fourth type of plate: a Costar 9102 γ-irradiated cell-culture grade plate. BSA, OVA, Hb and tPA were coated directly and wells were blocked with Roche Blocking Reagent and coating of proteins was visualized with the use of protein specific antibodies (FIG. 2K). In addition, wells were first blocked and protein was coated in carbonate buffer pH 9.6, afterwards. Strikingly, tPA is still able to bind to the wells of the ELISA plates, irrespective whether they were blocked or not. The other three proteins only bound to the unblocked plastic. This shows that tPA can bind to the Blocking Reagent. To further analyze this observation, we incubated blocked wells with a concentration series of the four proteins in PBS pH 7.3 (FIG. 2L). It can be clearly seen that tPA binds with high affinity to the Blocking Reagent, whereas the other proteins hardly bind. Calculated affinity constants Kd are 0.6 nM, undetermined, 195 nM and 98 nM for tPA, OVA, BSA and Hb, respectively. In order to determine whether this effect of inducing tPA binding sites in the Blocking Reagent was unique to the Costar 9102 plate, we analyzed tPA binding to the Blocking Reagent immobilized in wells of the Nunc Immobilizer Amino plate, the Greiner Microlon high-binding plate and the Costar 2595 plate (FIG. 2M). tPA bound again with high affinity to the Blocking Reagent at the surface of the Costar 9102 plate (Kd ˜0.5 nM). Affinities were 30 nM, 39 nM and 23 μM for the Costar 2595, the Nunc and the Greiner plate, respectively. In summary, tPA binds with high affinity to Blocking Reagent when the Blocking Reagent is bound to the wells of a Costar 9102 plate. From these combined observations, we conclude that polystyrene, vinyl, γ-irradiated polystyrene and polystyrene with coated organic spacers influence protein stability to various extents.

When the plasma protein lysozyme is exposed to the stainless steel of a Microlance-3 injection needle, tPA activating properties are induced (FIG. 2N). These observations indicate that lysozyme adopts a new conformation comprising cross-β structure upon exposure to the needle. A person skilled in the art easily expands these observations with other proteins including plasma or whole blood, and with various materials obtained from for example biomaterials, including stents, implants, catheders, heart pumps, dialysis membranes and tubings used for drawing of body fluids and in extracorporal circulations. In addition various materials and products that are being or can be used for the production and/or storage of proteins, preferably protein therapeutics, preferably for use in a mammal, can be tested. Not only ThT fluorescence and tPA activation can be examined, but also FXII activation, appearance under a TEM, binding of other cross-β structure binding compounds. Moreover, a person skilled in the art can test whether a compound or a combination of compounds can prevent the formation of cross-β structure formation by a given surface. Such a compound or compounds can be for instance non-amyloid peptides, for example FP10 or non-amyloid islet amyloid polypeptide of murine origin³. Alternatively the effect of coincubation with compounds that bind to compounds with cross-β structure, such as the compounds listed in Table 1-3 (prophylaxis) can be tested. In addition, coatings with single amino acids may prevent binding of proteins to surfaces, accompanied by cross-β structure formation.

We conclude that exposure of proteins to certain endogenous and non-self surfaces can induce cross-β structure and/or amyloid-like properties. The refolding of these proteins into a conformation comprising cross-β structure induces activation of tPA and FXII. These results disclose that problems, such as coagulation and inflammation, associated with the use of biomaterials are mediated by proteins comprising cross-β structure. In addition, the results presented herein disclose that problems, including immunogenicity, thrombotic complications, such as disseminated intravascular coagulation (DIC) or anaphylactic responses that are associated with the use of certain protein therapeutics are attributed to the induction of cross-β structure in said protein therapeutic or one of its constituents by contact with an artificial surface used for the production, storage or delivery of said therapeutic. Many if not all of the effects that are seen after introducing surfaces to the human body, e.g. inflammatory responses, activation of the plasma kinin forming cascade, historically known as the contact system of blood coagulation, complement activation, immune responses, are now attributed to the induction of protein conformations that are not present in the native molecules but induced upon contacting endogenous or foreign surfaces. Our data now indicate that these protein conformations comprise the amyloid-like cross-β structure conformation, the protein fold associated with various conformational diseases such as Alzheimer's-, Creutzfeldt-Jakob's- and Huntington's disease, but which is predicted to be able to be formed by virtually any polypeptide not necessarily associated yet with a disease. The cross-β structure conformation likely comprises the trigger of all of the observed responses of the body to certain surfaces. For example, tPA^{1,3} and FXII (data disclosed here) as well as a series of scavenger receptors, i.e. CD36, scavenger receptor A, scavenger receptor B-I, receptor for advanced glycation endproducts (¹ and references therein), and complement factor C1q^{25,26} are activated by cross-β structure rich polypeptides. Materials that can be tested for their ability to introduce the cross-β structure conformation in proteins are numerous. In implants, heart valves, heart aid devices, heart pumps, stents, slow release systems, extracorporal circulation devices and needled and tubings various materials are used, e.g. polyvinylchloride, stainless steel, polyamide, platinum, polypropylene, polytetrafluoroethylene, titanium, aluminium, tantalum, nickel, iridium and zirconium, to name a few.

Using a method of the present invention a person skilled in the art can select any medical device or implant, or material that is useful for the production of a medical device, implant or any other material for medical purpose that induces preferably no cross-β structure. Moreover, a person skilled in the art can now test the effect of any compound/condition/treatment on the formation of cross-β structure by said material or device. Preferably, said compound is coated on said material or device. Preferably said compound is FP10 or murine IAPP or any compound of table 1-3. A person skilled in the art can test the effect of said selected material on any of the aforementioned unwanted side effects caused by the present use of said materials or devices. For example, the effect of the material or device on the activity of tPA and FXII can be tested using the herein described tPA activation and factor XII activation assays. In addition, the effect on the adhesion of blood cells, including but not limited to platelets, neutrophils and lymphocytes can be determined. Preferably this is performed ex vivo with a device that is suitable to determine the adhesion under flow. In addition the effect of said material or device on platelet aggregation can be determined. Preferably this is also conducted under flow. In addition activation of the complement system can be determined. In vivo the effect of said materials can also be analyzed, for example a small disk of said material is implanted into a mouse. Subsequently recruitment of blood cells, preferably neutrophils and/or monocytes or macrophages is determined. In addition, any other signs of unwanted side effects, such as inflammatory responses, preferably the activation of factor XII and/or complement, cytokine release or any other sign of disease, such as fever and weight loss can be determined. Such analysis show further evidence that methods disclosed in the present application are suitable to design and/or improve the use materials for medical purposes.

### Example 2

### Exposure of Proteins to Various Factor XII Activating Surfaces Results in Enhancement of Factor XII/Kallikrein Activity, Indicative for Amyloid-Like Protein Misfolding

### Materials & Methods

### Factor XII/Prekallikrein Activation Assay Protocol

In previous examples we showed that exposure of albumin or endostatin in combination with factor XII to the known factor XII activating surfaces kaolin and DXS500k resulted in enhancement of factor XII/prekallikrein activation (see above). Furthermore, we established the denaturing capacity of these surfaces.

To provide further proof for the misfolded protein detection technology, we exposed a series of proteins to surfaces and analyzed the effect on the ability of the protein/surface mixtures to activate factor XII.

Immobilizer Polysorp plates (Nunc) were blocked with 200 μl PBS, 1% Tween20 for 1 hour. After blocking, the plates were rinsed twice with water. Ten μl of a surface solution was mixed with 10 μl of protein solution by pipetting, and individual surface or protein solutions were mixed with 10 μl buffer. All dilutions of proteins and surfaces were prepared in 1×HBS. Surfaces tested are kaolin, Ellagic acid (E2250, Sigma, St. Louis, Mo., USA) and lipopolysaccharide (LPS, L3024, Sigma, St. Louis, Mo., USA). Proteins tested are Bovine Serum Albumin (BSA; ICN #105033), Gelofusin (Braun Melsungen AG), Ovalbumin (A7641, Sigma, St. Louis, Mo., USA) and Endostatin (Entremed, Inc, Rockville, Md., USA). After mixing surfaces and proteins, plates were incubated under constant motion at room temperature for approximately 20 minutes. Twenty μl of chilled prekallikrein (PK) mix (2.5×HBS with 15 μM ZnCl₂, 2 μg/ml PK (Calbiochem), 2.1 μg/ml High Molecular Weight Kininogen (Calbiochem)) was added to each well. Subsequently, 10 μl of Chromozym-PK (Roche Diagnostics, Almere, The Netherlands, catalogue number 378445) was added to each well. To start the reaction, 10 μl of FXII mix was added to each well (HBS with 0.48 μg/ml factor XII, 5 μM ZnCl₂). Chromogenic substrate conversion was measured every minute in a spectrophotometer (Spectramax, Molecular Devices Ltd. Wokingham, England) for 3 hours at 37° C. Data was analysed by subtracting blank values, obtained at the beginning of the experiment, and all experiments were performed in duplicate. In single control wells, factor XII was omitted from the reaction mixture. No substrate conversion was seen with these controls (not shown).

Measurement of Amyloid-Like Protein Misfolding after Exposure of Proteins to (Bio)Medical Equipment

### Protein Solutions of Lysozyme, Ovalbumin, γ-Globulins and Albumin for Exposure to Surfaces.

Lysozyme from hen egg white (‘Lysoz’, Fluka BioChemika, 62971, Analysis Number: 52777/1 42497).
chicken egg Albumin (ovalbumin, OVA, Grade V: minimum 98%, Sigma, A-5503, Lot 14H7035).
γ-Globulins human, from Cohn Fraction II, III [9007-83-4], Sigma, G-4386, Lot 21K7600 (γ-glob).
Albumin, Bovine initial fractionation by heat shock, Fraction V, minimum 98%, Sigma, A-7906, Lot 56H0659 (BSA).
PBS buffer: “Fosfaat gebufferd zout” pH=7.4, apotheek UMC Utrecht, artikelnr.: 97907189, charge: 060501-009B2 (PBS).
Syringe used for sterilization: 10 ml BD Discardit II, 2011-02, Lot 0603 278.
Needle used for sterilization: 21G×1½″-Nr. 2, 0.8 mm×40 mm, BD Microlance, 2011-01, Lot 060219.
Filter used for sterilization of protein solutions: Sartorius, minisart, 0.20 μm, 16534, Lot 16534 060029.
15 ml tubes for protein solutions, poly-propylene-tube sterile, Greiner bio-one, 188271, Lot 06150196.
6.5% m/v sodium azide solution in H₂O
A. Polysulfone Dialyzer (Fresenius Medical Care AG, Bad Homburg, Germany)
B. Needle, Microlance-3 21G 1½″-Nr. 2, 0.8×40 mm, REF 304432 (Beckton Dickinson S.A., Fraga (Huesca), Spain)
C. 1 ml Glass vial with ppn screw cap (Omnilabo International, The Netherlands, catalogue number 260310)
D. sterile plastic tubing of the A1051 blood withdrawal system (Braun Melsungen AG, Melsungen, Hungary)

### Method: Protein Solutions

The lyophilized proteins, that were stored at 4° C., were dissolved in PBS to a concentration of 1 mg/ml in poly-propylene 15 ml tubes. To dissolve the proteins and allow the proteins to adopt a native conformation, a 10 minute incubation at a roller device at room temperature was followed by a 10 minute incubation at 37° C. in a incubator and again a 10 minute incubation at a roller device at room temperature. The protein solutions were pulled into a 10 ml syringe using a needle and filter-sterilized using Sartorius 0.2 μm filters, and kept at room temperature in a sterile 15 tube for approximately 5 hours, before storage at 4° C. (see below). Sodium azide is added to each solutions to a final concentration of 0.065% before subsequent use in analyses.

### Exposure of Proteins in Solution to Surfaces of (Bio)Medical Equipment: Analysis of Denaturing Capacity

Methods: Incubation of Proteins with Surfaces of (Bio)Medical Equipment

In order to analyze the protein denaturing potency of several products used routinely in the (bio)medical field, we incubated proteins at 1 mg/ml in poly-propylene tubes, filled with a product of interest or nothing (control). The control protein solutions are stored in the dark, still at 4° C. Equipment used is the polymer fibers of a polysulfone renal dialysis device (25 strands of approximately 8 cm), 10 needles used for injections, one glass vial with a plastic screw cap or eight pieces of approximately 1 cm of plastic tubing of a blood withdrawal system. To each surface, 1.5 ml of the 1 mg/ml protein solutions in PBS was added, to test the denaturing potency of the surfaces. Proteins incorporated in the analysis are BSA, OVA, lysozyme and human γ-globulins. As a control, surfaces are incubated solely with PBS. Tubes are fixed on a shaker, at 4° C. in the dark.

After 48 h of incubation, protein solutions and control PBS were analyzed for the capacity to enhance Congo red fluorescence and Thioflavin T fluorescence. For this purpose, 10 μl of the solutions was added to 90 μl PBS with 25 μM Congo red. Fluorescence at 550 nm after excitation at 595 nm was determined in black 96-wells plates, using a Thermo Fluoroskan Ascent 2.5 (Breda, The Netherlands). Thioflavin T fluorescence was determined by adding 10 μl sample to 90 μl of 50 mM glycine pH 9.0 with 25 μM Thioflavin T. Samples were analyzed in duplicate wells.

After 64 h of incubation, protein concentrations in supernatants were determined using a standard BCA kit (Pierce), and protein concentrations were adjusted accordingly to this assay. The protein solutions and PBS were analyzed for their potency to induce tPA/plasminogen activation. The protein solutions are analyzed after ten-fold dilution. The tPA and plasminogen concentrations are 400 pM and 20 μg/ml, respectively. The positive control in the activation assay is misfolded human γ-globulins, obtained after dissolving lyophilized γ-globulins in 1,1,1,3,3,3-hexafluoro-2-propanol and trifluoro-acetic acid, air-drying, dissolving in H₂O to 1 mg/ml and incubating at room temperature. Then, samples were kept at 4° C., still, in the dark.

After approximately 150 h from the start of the experiment, tPA/plasminogen activating properties of all protein solutions and PBS controls is analyzed.

### Example 2

### Results

### Detection of Protein Misfolding Upon Exposure to Various Factor XII Activating Surfaces

### Activation of Factor XII/Prekallikrein by Proteins Exposed to Surfaces

In a chromogenic factor XII/prekallikrein activation assay with active enzyme kallikrein read-out, the influence of 500 μg/ml Gelofusin in presence and absence of 100 μg/ml for Kaolin or 30 μg/ml for LPS was determined (FIG. 3A, B). Gelofusin alone does not stimulate factor XII/prekalikrein activation, whereas enhanced activity is seen when Gelofusin is mixed with kaolin or LPS. When BSA or endostatin in the presence or absence of 50 μg/ml Ellagic acid is tested in a chromogenic factor XII/prekallikrein activation assay, it is clearly seen that exposure of the proteins to the polyphenol surface induces increased capacity to activate factor XII/prekallikrein (FIG. 3C, D). Similar enhanced activation of factor XII/prekallikrein is observed when ovalbumin is exposed to kaolin, an effect that resembles our results with albumin and endostatin (see above).

Contacting DXS500k with various proteins, including lysozyme, γ-globulins, whole plasma and factor XII itself, results in the introduction of amyloid-like properties in the proteins, e.g. activation of tPA (FIG. 4A), enhanced fluorescence of ThT (FIG. 4B-D) and binding of tPA in an ELISA (FIG. 5A-D), demonstrating the formation of cross-β structure conformation in the protein aggregates after exposure to the negatively charged surface.

In summary, we show with the data depicted in FIG. 1 and FIG. 3-5 that exposure of various proteins to surfaces results in increased content of amyloid-like misfolded protein, as determined by assessing enhanced tPA binding, enhanced tPA activation, enhanced ThT fluorescence and enhanced factor XII activation.

Measurement of Amyloid-Like Protein Misfolding after Exposure of Proteins to (Bio)Medical Equipment

The protein denaturing potency of several surfaces of (bio)medical equipment was analyzed. We incubated the polymer fibers of a polysulfone renal dialysis membrane (25 strands of approximately 8 cm), 10 needles used for injections, one glass vial with a plastic screw cap or eight pieces of approximately 1 cm of plastic tubing of a blood withdrawal system with 1 mg/ml solutions of OVA, BSA, lysozyme or γ-globulins. In Table 4 the protein concentrations in all protein solutions is listed. For the tPA/plasminogen activation assay, for each protein concentrations of samples is adjusted to the lowest determined concentration by adding PBS. It is seen that all four proteins apparently bind to the polysulfone polymers, or that the proteins change conformation and thereby gain the ability to stick to the wall of the reaction vessel (15 ml poly-propylene tube). In addition, protein solution is depleted from the γ-globulins or lysozyme when both proteins are exposed to the glass vial and its plastic screw cap.

Upon exposure of OVA to the tubing of a plastic blood withdrawal system or to a glass vial including its plastic screw cap, fluorescence of Congo red is enhanced, indicative for misfolding of OVA accompanied by formation of amyloid-like structures (FIG. 6B).

When γ-globulins are exposed to tubes of a blood withdrawal system, needles used for injections or fibers of a renal dialysis membrane, the capacity to enhance Thioflavin T fluorescence is enhanced (FIG. 6C).

Exposure of lysozyme to needles used for injection or to a glass vial with its plastic screw cap induces increased activation of tPA/plasminogen, when compared to PBS control and lysozyme control (FIG. 6F). This is indicative for formation of tPA-activating amyloid-like protein conformation in the lysozyme, which is apparently present in solution and not solely at the surface of the materials.

In conclusion, we see that a number of proteins exposed to various materials that are used routinely in (bio)medical settings, adopt the amyloid-like misfolded protein conformation, resulting in enhanced Thioflavin T fluorescence, enhanced Congo red fluorescence, adsorption of protein to the reaction vessel wall or to the surface of the material, enhance activation of tPA/plasminogen, thereby exerting a fibrinolytic activity. Furthermore, a tPA/plasminogen activating substance and Congo red fluorescent enhancing factor is present in PBS buffer after exposure to polysulfone polymers used in renal dialysis devices.

TABLE 4
Protein concentrations after exposure to (bio)medical surfaces^{†}

| | concentration |
|---|---|
| Protein | (mg/ml) |
| BSA control in PBS | 1.232 |
| BSA polysulfone | 0.724 |
| BSA Glass vial | 1.268 |
| BSA Needles | 1.148 |
| BSA Tubing | 1.192 |
| OVA control in PBS | 0.940 |
| OVA polysulfone | 0.640 |
| OVA Glass vial | 0.988 |
| OVA Needles | 0.824 |
| OVA Tubing | 1.064 |
| γ-globulins control in PBS | 1.067 |
| γ-globulins polysulfone | 0.824 |
| γ-globulins Glass vial | 0.872 |
| γ-globulins Needles | 0.624 |
| γ-globulins Tubing | 0.920 |
| Lysozyme control in PBS | 0.860 |
| Lysozyme polysulfone | 0.616 |
| Lysozyme Glass vial | 0.856 |
| Lysozyme Needles | 0.668 |
| Lysozyme Tubing | 0.832 |

^{†}For all four proteins, 1 mg/ml solutions were prepared in PBS that were filter-sterilized using a 0.2 μm filter. This may explain protein concentrations of less than 1 mg/ml in control protein solutions.

### DESCRIPTION OF FIGURES

FIG. 1: Surfaces induce amyloid-like properties in various proteins.

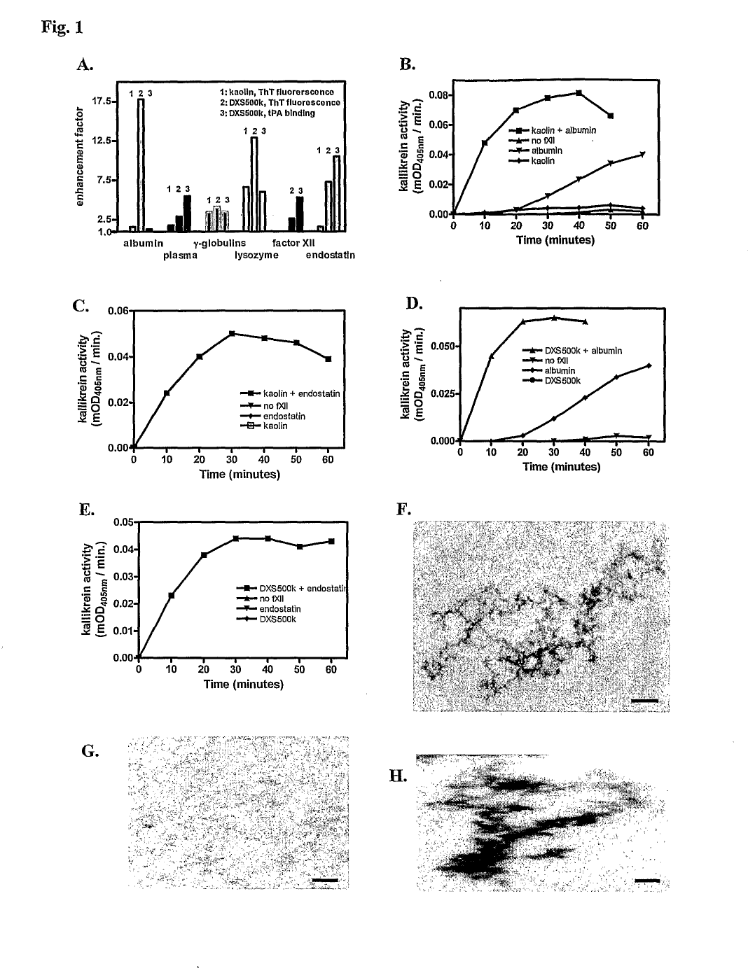

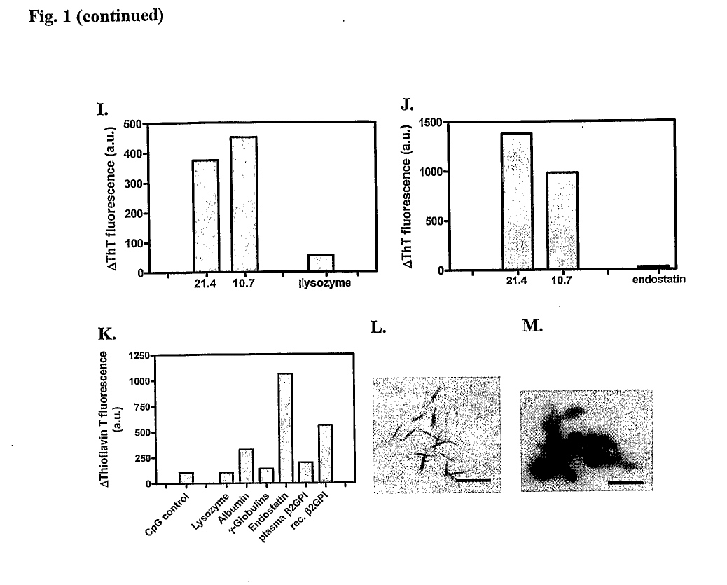

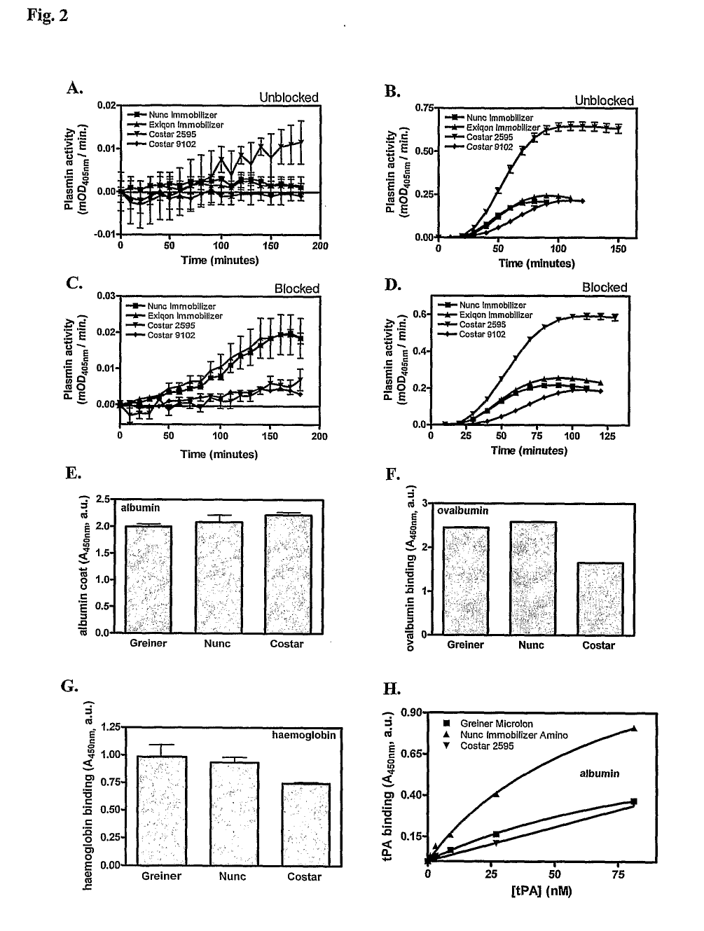

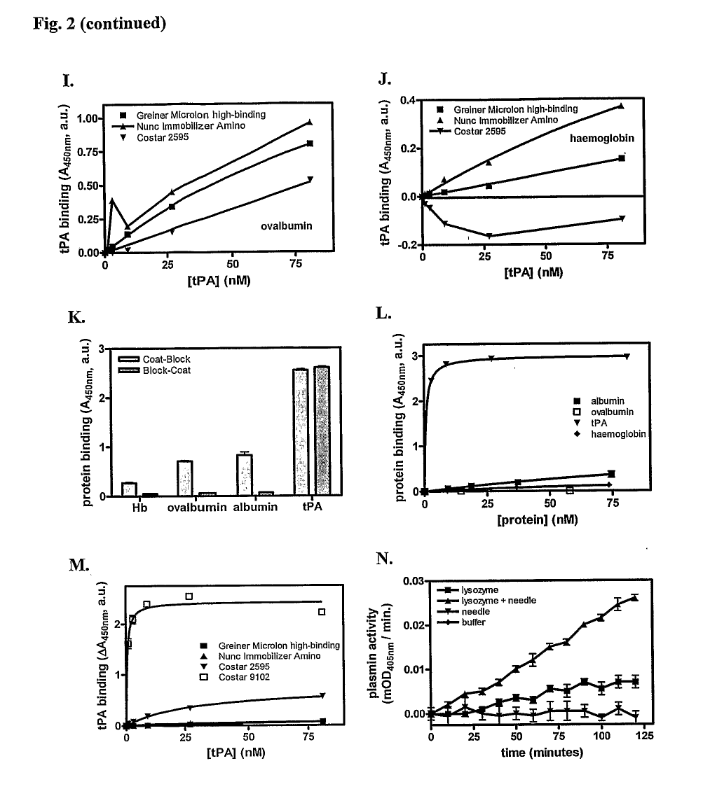

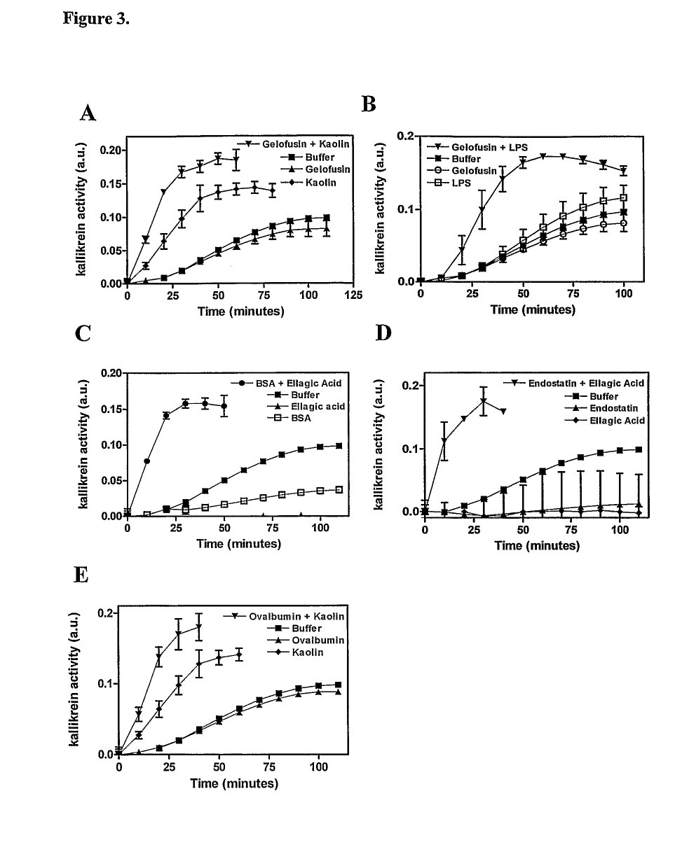

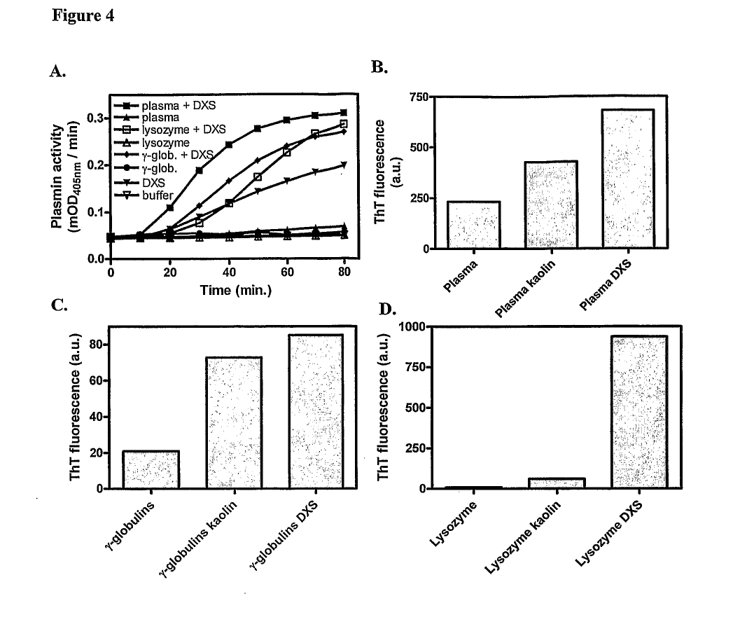

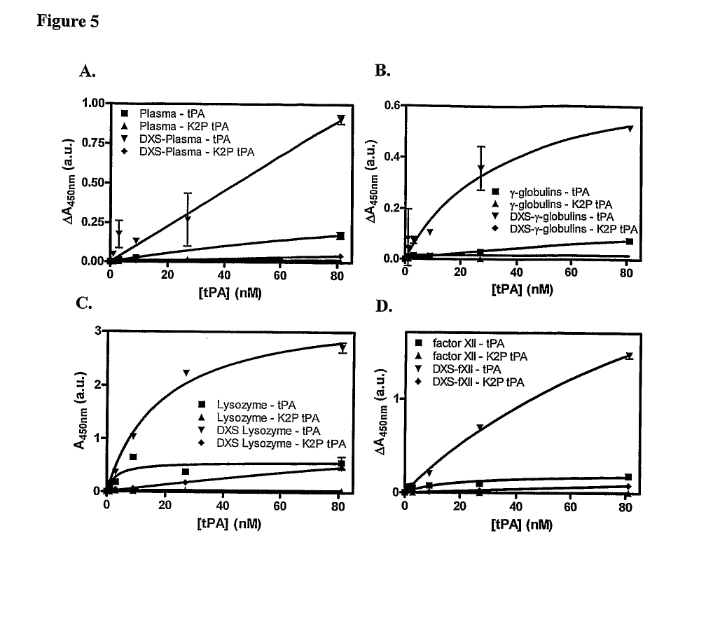

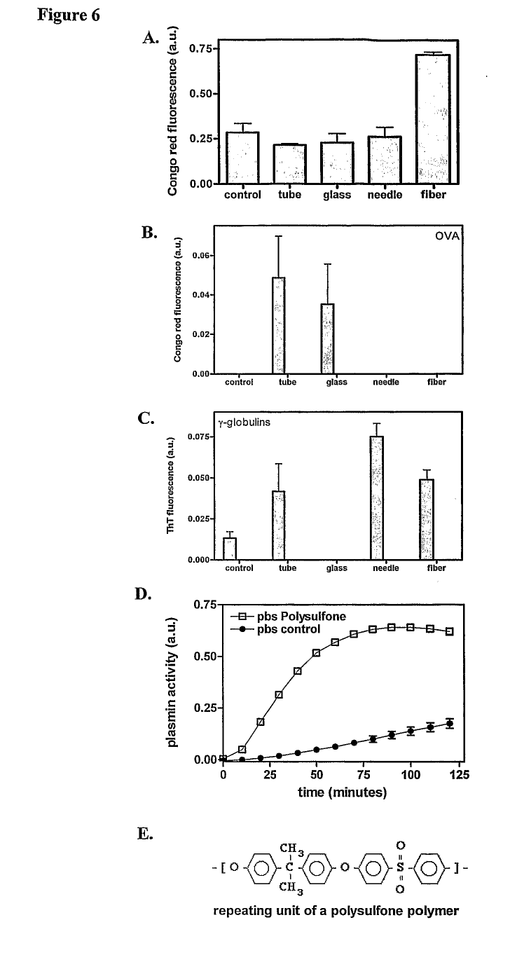

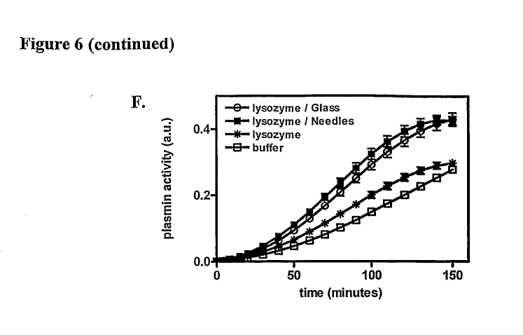

A. Polymer DXS500k and mineral kaolin induce ThT fluorescence, and polymer DXS500k induces tPA binding properties in various proteins after overnight incubation, as measured in an ELISA with immobilized proteins with or without DXS500k. ThT fluorescence or tPA binding with proteins incubated with DXS500k or kaolin is given as a multiple of the fluorescence or tPA binding observed when DXS500k and kaolin were omitted during protein incubations (‘enhancement factor’). B-E. FXII is only then effectively activated when both mineral particles of kaolin or polymer DXS500k and either 1 mg ml⁻¹ BSA (B., D.), or endostatin (C., E.) are included in the assay mix. Activation of FXII in the presence of prekallikrein and high molecular weight kininogen was determined by measuring conversion of chromogenic kallikrein substrate Chromozym-PK. F-H. TEM images of lysozyme (F.), DXS500k (G.), lysozyme exposed to DXS500k (H.). The scale bar represents 200 nm. I. Enhancement of ThT fluorescence as measured after a 30 minutes exposure of lysozyme to 21.4 and 10.7 μg ml⁻¹ CPG-ODN, at room temperature. J. Enhancement of ThT fluorescence as measured after a 30 minutes exposure of endostatin to 21.4 and 10.7 μg ml⁻¹ CPG-ODN, at room temperature. K. Exposure of 1 mg ml⁻¹ BSA, endostatin, plasma β2GPI or recombinant β2GPI to 21.4 μg ml⁻¹ CPG-ODN (overnight, at 4° C.) results in increased ThT fluorescence with approximately a factor 2 to 10. With these assay conditions no effect is seen with lysozyme and γ-globulins. L-M. TEM images of CpG-ODN (L.), and lysozyme exposed to CpG-ODN (M.). The scale bar represents 200 nm.

FIG. 2. Interaction of proteins with plastic surfaces.

A-D. A tPA activation assay is performed simultaneously in 8-well strips of four different 96-well plates, as indicated. Wells were either used directly (A., B.), or blocked with PBS containing 0.1% v/v Tween20 (Nunc Immobilizer and Exiqon Immobilizer, polystyrene with organic spacer) or with Roche blocking reagent (Costar 2595, vinyl and Costar 9120, γ-irradiated polystyrene) and washed twice with H₂O, prior to the assay (C., D.). Background activation of tPA and Plg was tested by omitting a cofactor with cross-β structure conformation (A., C.). The influence of an amyloid-like cofactor was tested by including 5 μg ml⁻¹ amyloid γ-globulins in the assays (B., D.). E-G. Analysis of coat efficiency on various types of ELISA plates by comparing binding of protein specific antibodies to BSA (E.), OVA (F.) and Hb (G.) immobilized onto Greiner Microlon high-binding- (Greiner), Nunc Immobilizer Amino- (Nunc) and Costar 2595 (Costar) 96-wells ELISA plates. Signals are used to calculate scale factors for signals obtained with tPA binding to the proteins coated onto the different ELISA plates. ELISA plates H-J. tPA ELISA with BSA (H.), OVA (I.) and Hb (J.) immobilized on a Greiner Microlon high-binding-, a Nunc Immobilizer Amino-, and a Costar 2595 vinyl plate. TPA binding to the proteins coated on the Greiner plate is set as a reference. Scale factors are determined from coat efficiencies derived by comparison of signals obtained with protein specific antibodies. K. ELISA showing the effect of Roche Blocking Reagent on the coat efficiency of Hb, OVA, BSA and tPA, as detected with protein specific antibodies. L. ELISA showing that tPA binds specifically and with high affinity to Roche Blocking Reagent immobilized on a Costar 9102 ELISA plate, when compared to OVA, BSA and Hb. M. ELISA showing the binding of tPA to Roche Blocking Reagent immobilized on four different types of ELISA plate. N. tPA activation assay showing that exposure of lysozyme to a Beckton-Dickinson Labware Microlance-3 needle introduces increased tPA activating properties.

FIG. 3. Detection of protein misfolding at surfaces by assessment of activation of factor XII and prekallikrein.

A., B. In a chromogenic factor XII/prekallikrein activation assay with active enzyme kallikrein read-out, the influence of 500 μg/ml Gelofusin in presence and absence of 100 μg/ml for Kaolin (A.) or 30 μg/ml for LPS (B.) is determined. C., D. One mg/ml BSA (C.) or endostatin (D.) in the presence and absence of 50 μg/ml Ellagic acid is tested in a chromogenic factor XII/prekallikrein activation assay. E. Activation of factor XII/prekallikrein by ovalbumin exposed to kaolin.

FIG. 4. Contacting various proteins and plasma to factor XII activating surfaces results in formation of amyloid-like protein conformation.

A. Contacting plasma, lysozyme and γ-globulins to DXS500k results in activation of tPA and plasminogen, as measured in the chromogenic tPA/plasminogen activation assay. DXS500k alone also results in some activation. Plasma, lysozyme or γ-globulins controls do not activate tPA and plasminogen. B. Overnight incubation at room temperature of plasma with kaolin or DXS500k results in increased fluorescence of amyloid dye ThT, when compared to incubation with buffer. C. Incubation of γ-globulins with kaolin or DXS500k also induces increased ThT fluorescence. D. DXS500k induces ThT fluorescence with lysozyme. Kaolin incubation results in a smaller increase in ThT fluorescence, when compared to buffer.

FIG. 5. Activation of tPA/plasminogen by proteins and plasma exposed to factor XII-activating surfaces.

A-D. In an ELISA set-up tPA binds specifically to plasma proteins (A), γ-globulins (B), lysozyme (C) and factor XII (D) that were pre-incubated overnight with DXS500k, whereas tPA does not bind to buffer-incubated proteins. K2P tPA that lacks the amyloid-like misfolded protein-binding F domain does not bind to surface-contacted proteins.

FIG. 6. Enhancement of Congo red or Thioflavin T fluorescence after exposure of proteins and PBS to surfaces.

Thioflavin T and Congo red fluorescence enhancement assays. Control: PBS or protein solutions stored in a poly-propylene tube. A. PBS was exposed to the polysulfone polymer fibers of a renal dialysis membrane, tubing of a blood withdrawal system, a glass vial or needles used for injections, for several days at 4° C., with motion. Congo red fluorescence was determined with a ten-fold diluted PBS sample. B. Congo red fluorescence is enhanced after incubation of ovalbumin with tubings of a blood withdrawal system and after exposure to a glass vial with its plastic screw cap. C. Thioflavin T fluorescence is enhanced with γ-globulins solutions that were exposed to tubings of a blood withdrawal system, needles used for injections or fibers of a renal dialysis membrane. D. PBS exposed for 64 h at 4° C. to polymer fibers of a polysulfone renal dialysis membrane induces tPA/plasminogen activation. E. Chemical structure of the monomer in polymer polysulfone fibers. F. Exposure of lysozyme to needles used for injection or to a glass vial with its plastic screw cap induces formation of tPA/plasminogen activating protein conformations, not seen in lysozyme control. Negative control: PBS buffer.

### REFERENCE LIST

1. Bouma, B. et al. Glycation induces formation of amyloid cross-beta structure in albumin. J. Biol. Chem. 278, 41810-41819 (2003).
2. Bucciantini, M. et al. Inherent toxicity of aggregates implies a common mechanism for protein misfolding diseases. Nature 416, 507-511 (2002).
3. Kranenburg, O. et al. Tissue-type plasminogen activator is a multiligand cross-beta structure receptor. Curr. Biol. 12, 1833-1839 (2002).
4. Collen, D., Lijnen, H. R. & Verstraete, M. Blood: Principles and practice of hematology. Handin, R. I., Lux, S. E. & Stossel, T. P. (eds.), pp. 1261-1288 (J.B. Lippincott Company, Philadelphia, 1995).
5. Silverberg, M., Reddigari, S. R. & Kaplan, A. P. Blood: Principles and practice of hematology. Handin, R. I., Lux, S. E. & Stossel, T. P. (eds.), pp. 1127-1150 (J.B. Lippincott Company, Philadelphia, 1995).
6. Townsend, K. P. et al. CD40 signaling regulates innate and adaptive activation of microglia in response to amyloid beta-peptide. Eur. J. Immunol. 35, 901-910 (2005).
7. Coraci, I. S. et al. CD36, a class B scavenger receptor, is expressed on microglia in Alzheimer's disease brains and can mediate production of reactive oxygen species in response to beta-amyloid fibrils. Am. J. Pathol. 160, 101-112 (2002).
8. El Khoury, J. B. et al. CD36 mediates the innate host response to beta-amyloid. J. Exp. Med. 197, 1657-1666 (2003).
9. Subang, R. et al. Phospholipid-bound beta 2-glycoprotein I induces the production of anti-phospholipid antibodies. J. Autoimmun. 15, 21-32 (2000).
10. Gebbink, M. F., Claessen, D., Bouma, B., Dijkhuizen, L. & Wosten, H. A. Amyloids—a functional coat for microorganisms. Nat. Rev. Microbiol. 3, 333-341 (2005).
11. Schousboe, I. Factor XIIa activation of plasminogen is enhanced by contact activating surfaces and Zn2+. Blood Coagul. Fibrinolysis 8, 97-104 (1997).

12. Braat, E. A., Dooijewaard, G. & Rijken, D. C. Fibrinolytic properties of activated FXII. Eur. J. Biochem. 263, 904-911 (1999).
13. Hoppener, J. W., Nieuwenhuis, M. G., Vroom, T. M., Ahren, B. & Lips, C. J. Role of islet amyloid in type 2 diabetes mellitus: consequence or cause? Mol. Cell. Endocrinol. 197, 205-212 (2002).
14. O'Nuallain, B. & Wetzel, R. Conformational Abs recognizing a generic amyloid fibril epitope. Proc. Natl. Acad. Sci. U.S. A 99, 1485-1490 (2002).
15. Kayed, R. et al. Common structure of soluble amyloid oligomers implies common mechanism of pathogenesis. Science 300, 486-489 (2003).
16. Schielen, J. G., Adams, H. P., Voskuilen, M., Tesser, G. J. & Nieuwenhuizen, W. Structural requirements of position A alpha-157 in fibrinogen for the fibrin-induced rate enhancement of the activation of plasminogen by tissue-type plasminogen activator. Biochem. J. 276, 655-659 (1991).
17. Hoppener, J. W., Ahren, B. & Lips, C. J. Islet amyloid and type 2 diabetes mellitus. N. Engl. J. Med. 343, 411-419 (2000).
18. Hoppener, J. W. et al. Extensive islet amyloid formation is induced by development of Type II diabetes mellitus and contributes to its progression: pathogenesis of diabetes in a mouse model. Diabetologia 42, 427-434 (1999).
19. Horbach, D. A., van Oort, E., Donders, R. C., Derksen, R. H. & de Groot, P. G. Lupus anticoagulant is the strongest risk factor for both venous and arterial thrombosis in patients with systemic lupus erythematosus. Comparison between different assays for the detection of antiphospholipid antibodies. Thromb. Haemost. 76, 916-924 (1996).
20. de Laat, B., Derksen, R. H., Urbanus, R. T. & de Groot, P. G. IgG antibodies that recognize epitope Gly40-Arg43 in domain I of {beta}2-glycoprotein I cause LAC and their presence correlates strongly with thrombosis. Blood., (2004).
21. Ryu, E. The efficacy of killed Trypanosoma evansi vaccines in mice. Zhonghua Min Guo. Wei Sheng Wu Xue. Za Zhi. 8, 280-285 (1975).
22. Shu, Q. et al. Antibody response in sheep following immunization with Streptococcus bovis in different adjuvants. Vet. Res. Commun. 25, 43-54 (2001).
23. Han, H. R. & Park, H. M. Effects of adjuvants on the immune response of staphylococcal alpha toxin and capsular polysaccharide (CPS) in rabbit. J. Vet. Med. Sci. 62, 237-241 (2000).
24. Watson, D. L. & Davies, H. I. Influence of adjuvants on the immune response of sheep to a novel Staphylococcus aureus vaccine. Vet. Microbiol. 34, 139-153 (1993).
25. Fan, R. & Tenner, A. J. Complement C1q expression induced by Abeta in rat hippocampal organotypic slice cultures. Exp. Neurol. 185, 241-253 (2004).
26. Sarvari, M. et al. Inhibition of C1q-beta-amyloid binding protects hippocampal cells against complement mediated toxicity. J. Neuroimmunol. 137, 12-18 (2003).

1. A method for determining a difference in the cross-β structure content of a protein in a reference sample compared to said protein in a test sample wherein the test sample has been subjected to a treatment that is expected to have an effect on the cross-β structure content of said protein, the method comprising:
determining in said reference sample the cross-β structure content of said protein
subjecting said protein to a treatment that is expected to have an effect on the cross-β structure content to obtain said test sample
determining in said obtained test sample the cross-β structure content of said protein
determining whether the cross-β structure content of the reference sample is different from the cross-β structure content in the test sample.

2. A method for selecting a treatment that essentially preserves the structure of a protein, the method comprising:
determining in a reference sample the cross-β structure content of said protein
subjecting said protein to a treatment that is expected to have an effect on the cross-β structure content to obtain a test sample
determining in said test sample the cross-β structure content of said protein
selecting the treatment that essentially preserves the structure of said protein.

3. The method according to claim 1, wherein said protein is a protein in a solution.

4. The method according to claim 3, wherein said protein in a solution is a body fluid, blood or a part thereof.

5. The method according to claim 1, wherein a mixture of different proteins is tested.

6. The method according to claim 1, wherein one particular protein and one particular treatment is tested.

7. The method according to claim 1, wherein at least one of said determining steps is performed with an enzymatic assay.

8. The method according to claim 1, wherein said treatment comprises a physical or mechanical treatment.

9. The method according to claim 1, wherein said treatment comprises a biochemical or chemical treatment.

10. The method according to claim 1, wherein said treatment comprises a physical or mechanical treatment and a biochemical or chemical treatment.

11. The method according to claim 8, wherein said physical or mechanical treatment comprises freezing or thawing or lyophilization of said protein or subjecting said protein to cold or heat or radiation such as X-rays, UV, IR, or subjecting said protein to pressure or air or vortexing or sonication or stirring or swirling or shaking or any combination thereof.

12. The method according to claim 9, wherein said biochemical or chemical treatment comprises subjecting said protein to water or high pH or low pH or to a buffer solution or to a liquid comprising a protein or to a liquid medium or to ion strength or to osmosis or to an organic or inorganic detergent or to a radical or contacting said protein with a solid surface, or any combination thereof.

13. The method according to claim 1, wherein said treatment comprising subjecting said protein to aging.

14. The method according to claim 12, wherein said solid surface is a metal or plastic or wooden or glass or cotton or silk surface or any combination thereof.

15. The method according to claim 2, wherein said treatment comprises contacting said protein with a biocompatible material.

16. The method according to claim 15, wherein said biocompatible material comprises coated biocompatible material.

17. The method according to claim 16, wherein said coated biocompatible material is coated with protein or peptide or amino acid.

18. A biocompatible part of the type made from a biocompatible material, wherein the improvement comprises:
using as a biocompatible material or coated biocompatible material, a biocompatible material obtainable by the method of claim 2 for preparing a biocompatible part.

19. The biocompatible part of claim 18, wherein said biocompatible part is a stent.

20. The biocompatible part of claim 18, wherein said biocompatible part is a part of an extracorporeal circulation device.

21. The method according to claim 2, wherein said treatment comprises contacting said protein with a material suitable for the interior of a bioreactor.

22. A process for preparing a bioreactor of the type made from a material suitable for the interior, wherein the improvement comprises:
using as a material suitable for the interior of a bioreactor obtainable by the method of claim 21 for preparing a bioreactor.

23. The method according to claim 2, wherein said treatment comprises contacting said protein with a material suitable for the interior of a storage device.

24. A process for preparing a storage device of the type having an interior material, wherein the improvement comprises:
using as a material obtainable by the method of claim 23 for preparing the storage device.
